(19) United States
(12) Patent Application Publication
(10) Pub. No.: US 2009/0148863 A1
(43) Pub. Date: Jun. 11, 2009

(54) Nanoparticle biosensors

(76) Inventors: Xiaohong Nancy Xu, Norfolk, VA (US); Tao Huang, Norfolk, VA (US); Prakash Nallathamby, Norfolk, VA (US)

Correspondence Address: FOLEY AND LARDNER LLP;SUITE 500, 3000 K STREET NW, WASHINGTON, DC, 20007, US

(21) Appl. No.: 12/232,184

(22) Filed: Sep. 11, 2008

Related U.S. Application Data
(60) Provisional application No. 60/991,548, filed on Nov. 30, 2007.
(60) Provisional application No. 61/059,639, filed on Jun. 6, 2008.

(51) Int. Cl. G01N 33/53

(52) U.S. Cl. 435/7.1

(57) ABSTRACT

Compositions which are useful in ultralow level of detection based on functionalized nanoparticles having exceptional combinations of properties including stability, brightness, binding specificity, and ability to be imaged at single nanoparticle resolution over desired period of time. The biological moieties on the nanoparticles preserve biological function. The nanoparticle surface can comprise a first monolayer component which is adapted to bind to a biological moiety. The biological moiety can be adapted to bind to an analyte. The nanoparticle surface can further comprise a second monolayer component, which is adapted to help expose the first monolayer component on the surface. Other components on the surface can help stabilize the nanoparticle. The nanoparticles are stable against aggregation, have photostability (non-photodecomposition and non-blinking), and can achieve single molecule detection in real time. Analytes can be detected at low levels both in solution and on surfaces such as cell surfaces.

### RELATED APPLICATIONS

This application claims priority to U.S. provisional application Ser. No. 60/991,548 filed Nov. 30, 2007 and also to U.S. provisional application Ser. No. 61/059,639 filed Jun. 6, 2008, which are each hereby incorporated by reference in their entireties.

### FEDERAL FUNDING STATEMENT

At least some work described herein was developed with use of federal government funding under NSF/NIRT grant BES 0507036, NIH grant RR15057-01, and NIH grant R01 GM0764401. The federal government has certain rights in the invention.

### BACKGROUND

References cited herein as endnotes are listed later in the application. No admission is made that any of these references are prior art.

A need exists to develop better detection methods, including biological detection methods, and in particular detection methods for analytes at particularly low concentrations or amounts. Also, methods are needed for detecting analytes both in solution but also surfaces including biological surfaces like cell membranes. Although progress has been made, more progress is needed, particularly reaching levels of single molecule detection (SMD) and single cell studies.

A wide variety of biological moieties need to be detected. For example, biological systems can respond to extremely low levels of various protein molecules (antigen, ligand, receptor).^{A1-A3} For instance, individual T-lymphocytes binding with ˜10 molecules of interleukin-2 can lead to the production of interleukin-1 and a cascade of other vital immune responses^{A4}. At such low concentrations (far below the dissociation constant), conventional analytical tools are unable to effectively detect these vital molecules. Furthermore, classical kinetic theories based upon the law of mass action are unlikely to be applicable to the study of binding mechanisms of individual protein molecules. Thus, a stochastic approach is required to describe the binding kinetics of individual protein-protein interactions. SMD offers the required detection sensitivity and stochastic measurement to solve such challenging problems.

Currently, the primary methods for the detection of protein molecules include UV-vis spectroscopy, immunohistochemistry, and radioactive detection assays (³H). Some of these methods require proteins to be isolated from bulk cells. Typically, these methods also involve several washing steps and hence are timeconsuming and cannot measure protein-protein interactions on living cells in real time. Recently, confocal fluorescence microscopy has been used to map proteins on living cells. However, fluorescence probes suffer photobleaching and single-fluorophor molecules exhibit blinking. Thus, high numbers of fluorescence dye molecules typically are needed to label antibodies in order to detect and characterize individual receptors on living cells for sufficient lengths of time.^{A5} Unfortunately, only limited numbers of the dye molecules can be conjugated with individual antibody molecules. Thus, a need remains to detect individual protein molecules on living cells and to follow their binding mechanisms for an extended period of time. Furthermore, many of these detection schemes are still based upon immunoenzymatic assay and cell-staining techniques, which includes several washing steps.

Recently, atomic force microscopy (AFM) has also been used to detect protein-protein interactions on solid surfaces (e.g., gold, mica) using force measurements.^{A6} Unlike smooth solid surfaces, the living cell surface is not rigid, which presents a challenge for force measurement. Furthermore, the ligand has to be immobilized on the AFM tip, and using the tip to locate the few receptors scattered on the living cell surface offers limited temporal resolution for measuring the binding rates.

Although noble metal nanoparticles (e.g., Au, Ag) randomly attached with antibodies using electrostatic interaction have been widely used as nanoparticle probes to detect antigens on cell surface using transmission electron microscopy (TEM)^{A7,8} cell death occurs under the vacuum of TEM. Thus, such methods cannot measure kinetics (binding rates) of protein-protein interactions on living cells. Recently, Ag and Au nanoparticles have been used for sensing proteins in solution^{A9-A12} and to study transport dynamics in living cells,^{A13-A17} showing the great potential of SMD.^{A15,17,18} A variety of approaches have been reported for SMD in solution^{A5,A17,A19-A23} and in living cells.^{A15,17,24-26} However, in particular, the study of specific interactions of individual protein-protein interactions on living cells still remains challenging.^{A17}

More specifically, particular types of proteins are becoming increasingly important for ultrasensitive detection. For example, cytokines are secreted regulatory proteins and play vital role in controlling cell survival, growth, differentiation, and function by binding with specific receptors and initiating immune regulation pathways.^{B1} At the cellular level, it takes only a few cytokines molecules to induce a significant cellular response,^{B2} underscoring the importance of developing new tools to image individual cytokine molecules and to characterize their functions in real time. Tumor necrosis factor-α (TNF-α), a 17 kDa protein, is a pro-inflammatory cytokine that can mediate a variety of biological effects, such as immune regulation, antitumor activity, viral replication, and infection reistance.^{B3, 4} Studies have shown that a variety of pathological conditions, including cancer, heart disease, diabetes and autoimmune diseases, led to overproduction of TNF-α, and adequate doses of TNF-α were vital to effectively treat diseases (e.g., cancer) without severe side effects.^{B3,4} Unfortunately, despite extensive research over decades, the underlying mechanisms about how TNF-α mediates these crucial biological functions still remain incompletely understood and it is thus important to develop ultrasensitive assays for accurate analysis of TNF-α. Several commercially available methods have been used for detecting TNF-α: enzyme-linked immunosorbent assays (ELISA),^{B5, 6} radioimmunoassay (RIA)^{B7} cytotoxicity assay^{B8}, flow cytometry,^{B9} and RT-PCR^{B10}. The ELISA method offers high sensitivity with a detection limit of 5 pg/mL.^{B5, 6} However, these conventional methods typically involve multiple staining and washing steps, which are time consuming and cannot be used in quantitative analysis of TNF-α in real time. Fluorescence quantum dots (QDs) protein microarray¹¹ and electrochemical immunoassay^{B12} have been developed to detect TNF-α. Nanoparticle-based detection scheme and sensors have also been reported for detecting other proteins, such as prostate-specific 4 antigen (PSA) and streptavidin in solution.^{B13-16} Unfortunately, these new techniques still cannot achieve real-time measurements and single-molecule detection sensitivity. Unlike bulk measurements, SMD offers the unique opportunity to investigate distinctive functions of individual molecules in real-time. Two primary criteria for successful detection of single molecules are (i) to design a detection volume in which a single molecule is present statistically; and (ii) to ensure sufficient high signal to noise ratio (S/N), allowing detection of single molecules.^{B17} A variety of approaches have been reported for SMD in solution and in living cells.^{B15, B17-19} Fluorescence microscopy and spectroscopy have been used as popular tools for SMD in solution and in living cells. Unfortunately, fluorescence probes (e.g., fluorophors, GFP, QDs) suffer photodecomposition, offering limited lifetime for probing dynamic events of interest and for quantitative analysis.^{B17, B19,B 20} Noble metal nanoparticles (Ag, Au) have unique optical properties, which depend on their size, shape, surrounding environment, and dielectric constant of the embedding medium.^{B21-23} Unlike fluorescent probes and QDs, these noble metal nanoparticles do not suffer photodecomposition and do not blink under dark-field optical illumination, and Ag nanoparticles process exceptionally high quantum yield of Raleigh scattering that are order magnitudes higher than fluorophors (e.g., R6G). Recently, the inventors have demonstrated the possibility of using these intrinsic optical properties of Ag nanoparticles for imaging single living cells in real-time with sub-100 nm spatial resolution and millisecond time resolution,^{B24-29} and for imaging single receptor molecules on single living cells.^{B24} Nonetheless, the inventors used low distribution of proteins on single living cells to control SMD volume and achieve SMD. Such detection schemes limited the possibility of realtime imaging and sensing individual protein molecules on single living cells where the expression level of individual proteins would vary among cells and over time. To use single nanoparticles as SMD volumes, one will need to prepare smaller stable Ag nanoparticles (<5 nm). Currently, it remained challenging to prepare small Ag nanoparticles (<5 nm) that are stable (non aggregation) in solution over time due to its high surface-area-to-volume ratio. Note that a variety of reported synthesis methods devoted to prepare size- and shape-controlled Ag nanoparticles beyond 10 nm in diameter.^{B27, 30, 31}

### SUMMARY

Provided herein are compositions, methods of making compositions, methods of using compositions, devices, instruments, and applications.

For example, one embodiment provides a composition comprising: at least one nanoparticle comprising a surface functionalized with at least one first monolayer component and at least one second monolayer component different from the first, wherein the molar ratio of the amount of the first monolayer component to the amount of the second monolayer component is adapted to provide a stability against aggregation of nanoparticles when a collection of the nanoparticles is dispersed in water.

Another embodiment provides a composition comprising: at least one noble metal nanoparticle comprising a surface functionalized with at least one first monolayer component and at least one second monolayer component different from the first, wherein the first monolayer component is further functionalized with a biological moiety, and wherein the molar ratio of the amount of the first monolayer component to the amount of the second monolayer component is adapted to provide a stability against aggregation of nanoparticles when a collection of nanoparticles is dispersed in water.

Another embodiment provides a composition comprising: at least one noble metal nanoparticle comprising a surface functionalized with at least one first monolayer component and at least one second monolayer component different from the first, wherein the first monolayer component is further functionalized with a biological moiety, and wherein the particle size is about 1 nm to about 5 nm, and wherein the molar ratio of the amount of the first monolayer component to the amount of the second monolayer component is adapted to provide a stability against aggregation of nanoparticles when a collection of the nanoparticles is dispersed in water.

Another embodiment provides a composition comprising: at least one noble metal nanoparticle comprising a surface functionalized with at least one first monolayer component and at least one second monolayer component different from the first, wherein the first monolayer component is further functionalized with a biological moiety, and wherein the composition comprises the nanoparticles having an average particle size of about 1 nm to about 10 nm.

Another embodiment provides a composition comprising: at least one nanoparticle comprising a surface functionalized with at least one first monolayer component and at least one second monolayer component different from the first, wherein the molar ratio of the amount of the first monolayer component to the amount of the second monolayer component is adapted to provide a stability against aggregation of nanoparticles when a collection of the nanoparticles is dispersed in water, wherein the first component is further functionalized with a biological moiety for binding to an analyte.

Another embodiment provides a method comprising: providing at least one noble metal nanoparticle, functionalizing the surface of the noble metal nanoparticle with a first monolayer component and a second monolayer component different from the first.

Another embodiment provides a method comprising: providing a composition comprising: at least one nanoparticle comprising a surface functionalized with at least one first monolayer component and at least one second monolayer component different from the first, wherein the first component is further functionalized with a biological moiety for binding to an analyte; and then binding the composition to an analyte.

Another embodiment provides a method of detecting an analyte using a sensor comprising a sensor molecule coupled to a nanoparticle, the method comprising: exposing the sensor to the analyte, whereby the sensor molecule selectively binds to the analyte; and detecting an optical property of the nanoparticle in real-time with single-molecule resolution.

Another embodiment provides at least one complex comprising a protein or protein fragment coupled to a nanoparticle, wherein the protein or protein fragment is bound to a protein or protein fragment on the surface of a biological cell, thereby coupling the nanoparticle to the biological cell; and further wherein the nanoparticle exhibits localized surface plasmon resonance.

Additional, more particular embodiments and advantages for at least some of these embodiments are also summarized.

More particularly, for example, in one or more embodiments described herein, the inventors have developed nonphotobleaching, nonblinking single Ag nanoparticle biosensors to quantitatively measure the binding kinetics and affinity of single protein molecules on single living cells for an extended period of time (hours) (FIG. 1), using single-nanoparticle optical microscopy and spectroscopy (SNOMS).^{A3,15,16,27} The Ag nanoparticles can be directly imaged based upon the high quantum yield (QY) of their Raleigh scattering, using a detection scheme with no need of fluorescence excitation, which effectively avoids autofluorescence of living cells and significantly improves the signal-to-noise ratio of SMD on single living cells.

In another particular embodiment, the inventors have fused a functional protein (ZZ, small portion of protein A (PrA), IgG-Fc binding protein, acting as a receptor for IgG) with a transmembrane domain, the translocation (T) domain of the diphtheria toxin, to prepare T-ZZ (e.g., Gillet et al. for context),^{A28-30} and anchored the T-ZZ onto living cell membrane to create the desired coverage of individual ZZ molecules on the living cell surface, mimicking individual antigen and receptor molecules on living cells (FIG. 1).

In this particular embodiment, the T domain includes 10 R-helices (TH1-9 and TH5′).^{A31} Helices TH8-9 are highly hydrophobic and are sandwiched and hidden from water by two layers of amphiphilic helices (TH1-4 and TH5-7). Thus, the T domain is fully soluble at neutral pH. When exposed to an acidic pH of ±5.0, the T domain changes its conformation as the three groups of helices come apart, leading to amphiphilic helices (TH1-7) floating at the hydrophobic/hydrophilic interface of the cellular membrane surface and the hydrophobic hairpin TH8-9 inserting into the hydrophobic lipid bilayer^{A28,32} (FIG. 1C). Thus, T domain anchors onto the surface of cell membrane at pH 4.8 and remains on the membrane surface as the pH is switched back to 7.4. Previous studies have demonstrated that T-ZZ retains its biological activity and binding affinity after being incubated at pH 4.8 for 80 min, and T-ZZ remains anchored on the living cell surface and does not internalize into the living cells.^{A28-30} With this approach in this embodiment, the inventors generate a clean and affordable model system to validate and optimize single-nanoparticle biosensors for detecting the binding affinity and kinetics of individual protein molecules on single living cells in real time. The approach using the T domain fusion protein as a pH-dependent membrane anchor is much simpler to quantitatively control the number of protein molecules on living cells than using molecular biology techniques to control the expression level of proteins on living cells. This approach also demonstrates the possibility of fabricating and altering a living cellular surface with functional receptors, offering new opportunities to engineer the cell surface without gene transfection, which have shown potential therapeutic applications (e.g., design of anticancer vaccines).^{A33}

These and other particular embodiments provide at least one or more of the following advances:

(i) design and synthesis of single metal nanoparticle such as, for example, silver nanoparticle biosensors that resist their aggregation in the buffer solution, retain their photostability, and preserve the bioactivity of conjugated protein molecules;

(ii) utilizing high QY of Rayleigh scattering of single Ag nanoparticle biosensors and a new detection scheme (SNOMS), the inventors effectively avoid autofluorescence of cells and photoinstability of fluorescent probes and achieve unprecedented sensitivity to quantitatively detect, map, and characterize binding affinity and kinetics of single receptor molecules on single living cells in real time over hours; and

(iii) using the unique properties of T domain and protein engineering approach, the inventors anchor functional receptors on the living cell surface, generating a new, clean model system for validation and calibration of tools, including for example nanoparticle biosensors, for probing single receptor molecules on single living cells in real time.

One can now use these tools for, for example, quantitative molecular imaging of single protein molecules on single living cells for a desired period of time, offering new methods for monitoring cascades of biochemical reactions in real time.

In addition, one can use single-nanoparticle biosensors and related detection scheme developed, for example, to quantitatively analyze single ligand (e.g., hIL-2) and single receptor molecules (e.g., hIL-2R) on single living cells for effective characterization of anticancer vaccines and for better understanding of their biological functions.

In accordance with other particular embodiments described herein, the inventors have developed non-photobleaching, non-blinking single-molecule nanoparticle optical biosensors (SMNOBS) for quantitative sensing and analysis of, for example, single TNF-α molecules and its binding reactions in real time. Unlike previous studies, in some of these embodiments, the inventors reduced the size of single Ag nanoparticles, for example, to 2.6 nm in diameter and controlled mole ratios of functional groups on the surface of nanoparticles, which allowed for a single monoclonal antibody (MAB) molecule to be attached onto a single nanoparticle and hence created a SMNOBS. The inventors found that the SMNOMS resisted photodecomposition and can be directly used for imaging and analysis of single TNF-α molecules and its binding reactions for long periods of time, e.g., hours.

In some additional particular embodiments, the inventors have successfully developed AgMMUA-MAB nanoparticles as SMNOBS to image and detect single TNF-α molecules and to measure their binding affinity and kinetics in real time. The inventors synthesized small stable Ag nanoparticles (e.g., 2.6±1.1 nm) and used small surface area of individual nanoparticles and its surface functional groups to control SMD. Using high dependence of LSPRS of single Ag nanoparticles on their surface functional molecules and high quantum yield of Raleigh scattering of Ag nanoparticles, the inventors detected single TNF-α molecules with unprecedented high sensitivity, selectivity and dynamic range (e.g., 0-200 ng/mL) and with no need of washing steps. In particular embodiments, the inventors showed that SMNOBS resisted photodecomposition and blinking, and could be used to quantitatively measure binding affinity and stochastic binding kinetics of single protein molecules (e.g., TNF-α-MAB) over hours in real time. A study showed that MAB conjugated with single AgMMUA nanoparticles retained its biological activities with KB of (1.7±0.1)×10⁶ M⁻¹. Such biosensors are well suited both for diagnosis of TNF-α related diseases and for probing their fundamental roles in a variety of biological functions. One can utilize the same approaches to provide other SMNOBS to image and characterize other single protein molecules (e.g., cytokines, antigens, and receptors) in real time for better understanding their roles in related biochemical pathways and disease diagnosis and therapy.

In particular embodiments, while the inventions described and claimed herein may not be limited by theory, the inventors have found in some embodiments that SN2 reaction, instead of SN1 reaction, can be used as a common reaction mechanism to successfully attach functional groups of interest onto nanoparticle surface without causing the aggregation of nanoparticles. The functionalized nanoparticles can be used to conjugate with protein molecules of interest. The attached proteins preserve their biological functions and activities. Thus, the functional nanoparticles can act as effective biosensors to sense and detect molecules of interest.

In other embodiments, the single nanoparticle biosensors also resist photodecomposition and photo-blinking and allow to be continuously monitored for any desired hours. Thus, it can be used to map individual protein molecules on single living cell surface in real-time, provide accurate measurements of critical parameters, such as binding affinity and binding rate of trace amount of key proteins on single living cells, and determine the dependent-parameters of binding affinity and kinetics, for a wide variety of applications, such as design of effective therapy (e.g., medicine and anticancer vaccines).

Single nanoparticle biosensors of present particular embodiments can possess sensitivity and selectivity at single molecule resolution with a dynamics range from 0 to 50 molecules. The unprecedented sensitivity and selectivity of single nanoparticle biosensors offer a new and unique single molecule assay that can meet the demand of a wide variety of applications of molecular analysis in vitro and in vivo, such as ultrasensitive detection of molecular markers for early disease diagnosis, for design of effective therapy (e.g. anticancer vaccine), for better understanding of basic biomedical mechanisms, as well as ultrasensitive analysis for effective environmental monitoring and homeland security.

In particular embodiments, the inventors have designed and developed reaction schemes that allow one to rationally synthesize single nanoparticle biosensors for quantitative molecular imaging and analysis of single protein molecules on single living cells for any desired period of time, offering new tools for single molecule sensing and imaging of analytes of interest for earlier disease diagnosis, design and characterization of effective therapy (e.g., drug screening, anticancer vaccine), probing molecular mechanisms of biochemical reactions, and ultrasensitive environmental monitoring and homeland security.

In particular, provided herein is a nanoparticle sensor that resists aggregation in the buffer solution, retains their photostability, preserves the bioactivity of conjugated protein molecules, and shows its sensitivity, selectivity and a wide dynamic range at the single-molecule level.

Other advantages for one or more embodiments described herein include, for example: sensitivity and selectivity at single molecule level; dynamic range at 0-50 molecules per cell; lifetime: dependent upon the lifetime of conjugated protein; non-photo-decomposition and non-blinking (resist photodecomposition and blinking); offering the highest quantum yield (QY) with single nanoparticle resolution; serving as multiple color probes for continuously mapping and imaging multiple single protein molecules on single living cells at nanometer spatial resolution in real-time for any desired time; no need for expensive excitation source and can use inexpensive common halogen lamp; easy to synthesize and purify with low cost; long lifetime including stable for months.

In summary of some of these embodiments, at the cellular level, a small number of protein molecules (receptors) can induce significant cellular responses, emphasizing the importance of molecular detection of trace amounts of protein on single living cells. In accordance with the present invention, the inventors designed and synthesized silver nanoparticle biosensors (AgMMUA-IgG) by functionalizing 11.6±3.5-nm Ag nanoparticles with a mixed monolayer of 11-mercaptoundecanoic acid (MUA) and 6-mercapto-1-hexanol (1:3 mole ratio) and covalently conjugating IgG with MUA on the nanoparticle surface. The inventors found that the nanoparticle biosensors preserve their biological activity and photostability and can be utilized to quantitatively detect individual receptor molecules (T-ZZ), map the distribution of receptors (0.21-0.37 molecule/μm²), and measure their binding affinity and kinetics at concentrations below their dissociation constant on single living cells in real time over hours. The dynamic range of detection of this biosensor is 0-50 molecules per cell. The inventors also found that the binding rate (2-27 molecules/min) is highly dependent upon the coverage of receptors on living cells and their ligand concentration. The binding association and dissociation rate constants and affinity constant are k₁=(9.0±2.6)×10³ M⁻¹ s⁻¹, k₁=(3.0±0.4)×10⁻⁴ s⁻¹, and K_{B}=(4.3±1.1)×10⁷ M⁻¹, respectively.

Also, in accordance with the present invention, the inventors report here the synthesis of small stable silver nanoparticles (2.6±1.1 nm) and use its small surface area and monolayer of mixed functional groups, to control single molecule detection volumes on the surface of single nanoparticles. These new approaches allowed us to develop intrinsic single molecule nanoparticle optical biosensors (SMNOBS) for sensing and imaging of single human cytokine molecules, recombinant human tumor necrosis factor-α (TNFα), and probing its binding reaction with single monoclonal antibody (MAB) molecules in real-time. The inventors found that MAB covalently conjugated with a functional group on single Ag nanoparticles retained their biological activity. SMNOBS showed exceptionally high photostability (nonphotodecomposition and non-blinking over 12 h). The inventors utilized localized surface Plasmon resonance spectra (LSPRS) of SMNOBS to sense and image single TNFα molecules in solution, and to monitor the binding reaction of single TNFα molecules with single MAB molecules on the surface of single nanoparticles in real time, which showed a red shift of peak wavelength of 29±11 nm and stochastic binding kinetics of single molecule reactions with binding equilibrium times ranging from 30 to 120 min. The inventors used the SMNOBS to measure the binding affinity (K_{B}) of TNFα with MAB as (1.7±0.1)×10⁶ M⁻¹. SMNOBS exhibited extraordinarily high sensitivity, selectivity and dynamic range of 0-200 ng/mL, which is well suited for the fundamental study of biological functions of single TNFα molecules and development of effective disease diagnosis and therapy.

### BRIEF DESCRIPTION OF THE DRAWINGS

The patent or application file contains at least one drawing executed in color. Copies of this patent or patent application publication with color drawing(s) will be provided by the Office upon request and payment of necessary fee.

FIG. 1: Schematic illustration of preparation of single-nanoparticle biosensors (AgMMUA-IgG) and using the biosensors to image and detect single T-ZZ molecules on single living cells. (A) Functionalizing Ag nanoparticles with a mix monolayer of MUA and MCH (1:3 mole ratio) by strong interaction of Ag with thiol group of MUA and MCH. (B) Covalently linking AgMMUA nanoparticles with IgG via a peptide bond using EDC and sulfo-NHS as mediators, to prepare AgMMUA-IgG nanoparticles. (C) Attaching T-ZZ onto living cells by adjusting pH to 4.8 and detecting single T-ZZ molecules on single living cells using single-nanoparticle biosensors.

FIG. 2: Characterization of optical properties of Ag, AgMMUA, and AgMMUA-IgG nanoparticles. (A) Representative UV-vis spectra of (a) 3.28 nM Ag and (b) 4.33 nM AgMMUA nanoparticles in Nanopure water and (c) 5.24 nM AgMMUA-IgG nanoparticles in PBS buffer. (B) LSPR spectra of the representative single Ag, AgMMUA, and AgMMUA-IgG nanoparticle in the solution of (A). (C) Histograms of color distributions of single Ag, AgMMUA, and AgMMUA-IgG nanoparticles in the solution of (A).

FIG. 3: Characterization of biological activity of AgMMUA-IgG nanoparticles. (A) UV-vis spectra of 250 μL of 1.1 nM AgMMUA-IgG in the PBS buffer solution (a) before, at (b) 5 min, and at (c) 24 h after adding 1.0 μL of 25 μM PrA, showing a decrease in absorbance. (B) Plot of peak absorbance of the solution corrected with baseline in (A) versus the incubation time, showing the exponential decay: points are experimental measurements and solid line is generated by fitting the data points with a equation, Abs=0.23+0.059e^{(-0.0039 t)} and R²=1.0. (C) Plots of peak absorbance in (B) versus the incubation time at the early reaction time (0-90 min), showing a linearity with a slope of −2.0×10⁻⁴ min⁻¹, intercept of 0.29, and R²=0.99. R² in (B) and (C) is the multiple correlation coefficient and linear regression coefficient of determination, respectively.

FIG. 4: Imaging and sensing of single T-ZZ molecules on single living cells using single AgMMUA-IgG nanoparticle biosensors. The living cells attached with T-ZZ (0.37 molecules/μm²) were incubated with 5.56 nM AgMMUA-IgG nanoparticles and imaged using our SNOMS imaging system. The scale bar is 10 μm.

FIG. 5: Imaging and characterization of the concentration dependence of binding kinetics of AgMMUA-IgG with T-ZZ on single living cells at the single-molecule resolution. Sequence optical images of representative cells selected from at least 120 cells at each given reaction time (0-180 min) and concentration. (A) Coverage of T-ZZ on living cells (0.37 molecules/μm²) remains constant, which are incubated with (a) 1.39, (b) 2.78, (c) 5.56, and (d) 11.12 nM AgMMUA-IgG nanoparticles, showing the time- and concentration-dependence. (B) AgMMUA-IgG nanoparticle concentration remains constant at 2.78 nM, which is used to image the cells with the coverage of T-ZZ at (a) 0.21 and (b) 0.37 molecules/μm². (C) Control experiments were carried out using 11.12 nM AgMMUA, instead of AgMMUA-IgG to image the cells with coverage of T-ZZ at (a) 0.21 and (b) 0.37 molecules/μm². (c) Incubation of 11.12 nM AgMMUA-IgG nanoparticles with the cells that were not attached with T-ZZ. The scale bar is 10 μm.

FIG. 6: Characterization of binding affinity and kinetics of AgMMUA-IgG with T-ZZ on living cells and determination of dynamic range of single nanoparticle biosensors. Plots of number of single T-ZZ molecules bound with AgMMUA-IgG nanoparticles on single living cells, acquired from images similar to those shown in FIG. 5, versus incubation time. (A) Four concentrations of AgMMUA-IgG are used: (a) 1.39, (b) 2.78, (c) 5.56, and (d) 11.12 nM, as described in FIG. 5A. (B) The coverage of T-ZZ on living cells: (a) 0.21 and (b) 0.37 molecules/μm2, which were prepared as described in FIG. 5B. (C) Control experiments: (A-C) as described in FIG. 5C.

FIG. 7: Dependence of binding rates and fraction (f) of T-ZZ molecules bound with AgMMUA-IgG on living cells upon the concentration of AgMMUA-IgG. (A) Plot of binding rates calculated from FIG. 6A during 0-60-min reaction time (prior to equilibrium) versus concentration of AgMMUA-IgG nanoparticles. (B) the fraction (f) of bound T-ZZ molecules on living cells from FIG. 6A during 80-120-min reaction time (at equilibrium) versus concentration of AgMMUA-IgG (isotherms). The fraction (f) is calculated by dividing the number of T-ZZ molecules bound with AgMMUA-IgG nanoparticles on 40 living cells with the total number of T-ZZ molecules on the living cells.

FIG. 8: Representative HR TEM images of Ag nanoparticles, showing the size and shape of single Ag nanoparticles. Scale bar=10 nm. Histogram of 100 nanoparticles characterized by TEM shows that the size of nanoparticles is 11.6±3.5 nm.

FIG. 9: (A) Representative NMR spectrum of AgMMUA nanoparticle; (B) zoom in of individual peaks of NMR spectrum in (A): (a) δ=1.25, integration=4.50, (6H, HSCH₂—(CH₂)₃— of MUA); δ=1.36, integration=10.91, (4H, HSCH₂—(CH₂)₂— of MCH); δ=1.52, integration=8.19, (4H, —CH₂)₂—CH₂OH of MCH); and δ=1.69, integration=6.62, (10H, —(CH₂)₅—CH₂COOH of MUA). (b) δ=2.32 ppm, integration=1.83, (2H, —(CH₂)—COOH of MUA). (c) δ=2.55 ppm is from the —CH₂— next to thiol from both MUA and MCH and 4H from citrate with the integration of 119. (d) δ=3.55 ppm, integration=5.65, (2H, —(CH₂)—OH of MCH). The result showed the mole ratio of MUA to MCH=1:3.

FIG. 10: Schematic illustration of synthesis of photostable SMNOBS (AgMMUA-MAB) for sensing single TNF-α molecules:

(A) Functionalizing Ag nanoparticles with monolayer of mixed MUA and MCH using the interaction of the thiol group (—SH) with the surface of Ag nanoparticles to prepare AgMMUA;
(B) Linking the amine group of a MAB with the carboxyl group of the MUA attached on the nanoparticles via a peptide bond using EDC and sulfo-NHS as mediators to prepare AgMMUA-MAB nanoparticles.

FIG. 11: Characterization of size and photostability of single Ag nanoparticles:

(A) HRTEM image and (B) histogram of size distribution of Ag nanoparticles, showing the average diameter of Ag nanoparticles at 2.6±1.0 nm. The scale bar is 10 nm. (C) Dark-field optical image of single Ag nanoparticles, showing two representative colors, (a) blue and (b) green of Ag nanoparticles, and (c) background, as squared, respectively. (D) Plots of scattering intensity of (a) single green nanoparticle, (b) single blue nanoparticle, and (c) background in (C) versus illumination time, exhibiting that single Ag nanoparticles resist photodecomposition and blinking.

FIG. 12: NMR characterization of functional groups of AgMMUA nanoparticles:

(A) Representative NMR spectra of AgMMUA nanoparticles; (B) Zoom-in of individual peaks of NMR spectra in (A): (a) δ=3.55 ppm, integration=2.00, (2H, —(CH₂)—OH of MCH); (b) δ=3.18 ppm, integration=8.65, (2H, from PVP ring); (c) δ=2.55 ppm, integration=25.23, (—CH₂— next to thiol from both MUA and MCH and 4H from citrate. (d) δ=1.20-1.45 ppm, integration=6.05, (12H from —(CH₂)₆—CH₂COOH of MUA and 4H from —(CH₂)₂—CH₂OH of MCH). Molar ratio of MCH:MUA:PVP:Citrate=28:5:1:0.04.

FIG. 13: Characterization of optical properties of single Ag, AgMMUA and AgMMUA-MAB nanoparticles:

(A) UV-Vis spectra of 300 nM (a) 2.6 nm Ag nanoparticles in nanopure water; (b) AgMMUA in nanopure water; (c) AgMMUA-MAB in 10 mM PBS (pH 7.0), showing that the peak wavelengths at 388 nm (FWHM=66 nm; A=1.42), 406 nm (FWHM=116 nm; A=1.14), and 410 nm (FWHM=138 nm; A=0.74), respectively.
(B) Histograms of color distribution of single nanoparticles in (A) 2.6 nm Ag nanoparticle in nanopure water: (82±2) % of blue and (18±2) % green nanoparticles; (b) AgMMUA in nanopure water: (17±2) % of blue and (83±2) % green nanoparticles; and (c) AgMMUA-MAB in 10 mM PBS (pH 7.0): (3±1) % of blue and (97±1) % green nanoparticles.
(C) LSPR spectra of a representative single blue (a) Ag nanoparticle (λₘₐₓ=465 nm; FWHM=53 nm), (b) AgMMUA nanoparticle (λₘₐₓ=474 nm; FWHM=63 nm), and (c) AgMMUA-MAB nanoparticle (λₘₐₓ=477 nm; FWHM=74 nm);
(D) LSPR spectra of a representative single green (a) Ag nanoparticle (λₘₐₓ=513 nm; FWHM=64 nm), (b) AgMMUA nanoparticle (λₘₐₓ=543 nm; FWHM=69 nm), and (c) AgMMUA-MAB nanoparticle (λₘₐₓ=570 nm; FWHM=76 nm).

FIG. 14: Characterization of bioactivity and dynamic range of AgMMUA-MAB nanoparticles (SMNOBS) with TNF-α molecules:

(A) UV-vis spectra of 50 nM AgMMUA-MAB nanoparticles incubated with 500 nM TNF-α in the PBS buffer solution at (a) 0 min, (b) 20 min and (c) 24 hr, showing peak wavelength of 417±3 nm and a decease in absorbance over time;
(B) Plot of peak absorbance of the solution corrected with baseline in (A) versus the incubation time, showing the exponential decay: points are experimental measurements and a solid line is generated by fitting the data points with a equation, A=0.13+0.11e^{-0.011t} and R²=0.98;
(C) Plot of peak absorbance in (B) versus the incubation time at the early reaction time (0-40 min), showing a linearity with a slope of −8.9×10⁴ min⁻¹, intercept of 0.23, and R²=0.99. R² in
(B) and (C) is the multiple correlation coefficient and linear regression coefficient of determination, respectively.
(D) Calibration curve of AgMMUA-MAB nanoparticles for sensing TNF-α Plot of decreases in absorbance of AgMMUA-MAB nanoparticles (AA) versus TNF-α concentrations, showing the dynamic range of SMNOBS at 0-200 ng/mL with a slope of 1.2×10⁻⁴ mL/ng and R²=0.99.

FIG. 15: Real-time sensing of single TNF-α molecules using SMNOBS and probing binding reactions of single protein molecules (TNF-α with MAB):

(A) Snap shots of real-time optical images of single AgMMUA-MAB nanoparticles incubated with TNF-α at (a) 0, (b) 10, (c) 20, (d) 30, (e) 60, and (f) 240 min;
(B) LSPR spectra of a representative single AgMMUA-MAB nanoparticle circled in (A) incubated with TNF-α at (a)-(f) (0-240 min), showing the red shift of peak wavelength of 29 nm over 30 min of incubation time;
(C) Plots of peak wavelength (λₘₐₓ) of single nanoparticles in (A) versus incubation time, showing stochastic binding equilibrium times at: (i-ii) 30, (iii) 120, (vi) 30-60, (v)120, and (vi) 30 min, respectively.

FIG. 16: Characterization of selectivity and specificity of SMNOBS (control experiment): (A) Snap shots of real-time optical images of single AgMMUA-MAB nanoparticles incubated with BSA at (a) 0 and (b) 240 min, showing that the color of single nanoparticles remained unchanged over time; (B) LSPR spectra of a representative single AgMMUA-MAB nanoparticle squared in (A), incubated with BSA at (a) 0 and (b) 240 min, showing LSPRS remained unchanged over 8 hr.

### DETAILED DESCRIPTION

### Introduction

All references cited herein are incorporated by reference in their entireties. No admission is made that any of these references are prior art.

Priority U.S. provisional application Ser. No. 60/991,548 filed Nov. 30, 2007 and also U.S. provisional application Ser. No. 61/059,639 filed Jun. 6, 2008 are each hereby incorporated by reference in their entireties including the working examples, description, figures, and claims, including description and claims for biosensors.

Copending patent application to Xu et al. Ser. No. 12/219,223 filed Jul. 17, 2008, and also Nallathamby, et al., ACS Nano 2008, Vol. 2, No. 7, 1371-1380, are hereby incorporated by reference in their entirety including figures, claims, description, and working examples. In particular, this application describes washing and purification methods which can improve nanoparticle stability and purity of dispersion of nanoparticles.

The paper, “Design and Synthesis of Single-Nanoparticle Optical Biosensors for Imaging and Characterization of Single Receptor Molecules on Single Living Cells,” by Huang et al, Analytical Chemistry, 2007, 79, 7708-7718, is hereby incorporated by reference in its entirety.

Various elements are further described. The working examples provided below provide particular embodiments for these elements.

### Nanoparticle

A nanoparticle and collections of nanoparticles are known in the art including colloidal forms of nanoparticles. Nanoparticles can be dispersed in a solvent as a solution or as a dispersion or colloidal form. Nanoparticles in the context of ultrasensitive bioanalysis and nanoscale technology in biological systems are known in the art. See for example, Xu (Ed.), New Frontiers in Ultrasensitive Bioanalysis, Wiley, 2007; Mirkin, Niemeyer (Eds.), Nanobiotechnology II, Wiley, 2007; Greco, Rinz, Smith (Eds.) Nanoscale Technology in Biological Systems, (including Chapter 3, and description of use of nanoparticles in biological assays, drug delivery vehicles, and contrast agents). See also Poole, Owens, Introduction to Nanotechnology, Wiley, 2003, including chapters on nanoparticles, particle size measurement, self assembly, monolayers, and biological materials.

The shape of the nanoparticle is not particularly limited but can be approximately spherical or can have some elongation. In one embodiment, the nanoparticle is not triangular in shape as described in, for example, Haes et al., J. Phys. Chem. B, 2005, 109, 11158-11162, or Haes et al., J. Am. Chem. Soc., 2002, 124, 10596-10604.

The nanoparticle can be an inorganic nanoparticle, including an inorganic inner structure, but can also comprise an organic outer layer. In an embodiment, the nanoparticle is not magnetic.

The nanoparticle can be a metal nanoparticle or also can be a noble metal nanoparticle wherein most or essentially all of the nanoparticle material is a metal or noble metal. Examples include silver or gold. In one embodiment, the nanoparticle consists essentially of noble metal so that components which materially detract from advantages described herein are not compromised.

The size of the nanoparticle can be for example on average about 1 nm to about 100 nm, or about 2 nm to about 50 nm, or about 3 nm to about 25 nm. In one embodiment, the average size of the nanoparticle can be relatively small such as for example about 0.5 nm to about 10 nm or about 1 nm to about 5 nm. The size can be estimated as a diameter of particle. Size can be for an individual nanoparticle or a collection of nanoparticles, and statistical methods known in the art can be used, including measurements of monodispersity, standard deviations, and determination of percentages of particles which fall within a size range.

The nanoparticle can be free from binding to a solid surface. The nanoparticle can be unattached relative to a solid surface. The nanoparticle can be free to diffuse around in solution or dispersion. Nanoparticles can be dispersible and can be subject to separation methods such as centrifugation. In other embodiments, however, the nanoparticle can be bound to a solid surface.

The nanoparticle can comprise a uniform particle structure or can comprise a core-shell structure or layered structure.

One of skill in the art will be familiar with numerous methods to characterize nanoparticles, including composition and optical properties and size, including methods cited in the working examples and references herein and below. Methods related to real time imaging and single molecule detection are particularly of use. Examples of techniques that can be used to characterize nanoparticles include for example using a transmission electron microscope (TEM), dynamic light scattering, UV-visible spectroscopy, dark-field single nanoparticle optics microscopy and spectroscopy (NSOMS), and/or energy dispersion spectra (EDX) for the particle size and elemental composition.

### Nanoparticle Surface and Functionalization

Nanoparticle surfaces are known in the art and can be characterized by methods known in the art. The surface of the nanoparticle can be the surface of a core of the nanoparticle. Surface analysis in the biological context is known in the art. See, for example, Synthesis, Functionalization and Surface Treatment of Nanoparticles, by Marie-Isabelle Baraton (Editor); Andrade (Ed.), Surface and Interfacial Aspects of Biomedical Polymers, Plenum, 1985.

The surface of the nanoparticle can be functionalized as known in the art. In particular, the nanoparticle can be functionalized to convert it from a relatively unreactive form to a form that will be reactive, particularly in biological environments and show specific binding. The nanoparticles can be functionalized to show improved stability.

### Monolayer

A monolayer is known in the art including the disposition of a monolayer on a particle surface including a nanoparticle surface. See, for example, Love et al., Chem. Rev., 2005, 105, 1103-1169. Monolayers can form by self-assembly and self-assembled monolayers are known. See for example Terrill et al., J. Am. Chem. Soc., 1995, 117, 12537-12548; Hostetler et al., Langmuir, 1996, 12, 3604-3612. The monolayer can comprise at least one or more than one components which are different from each other and can serve different purposes or functions in the use of the nanoparticle. For example, the monolayer can comprise a first component, and a second component, and a third component, and higher numbers of components.

A mixed monolayer can be a monolayer system which comprises at least two components, each different from the other and providing a different function to the particle.

The monolayer components may undergo some sort of a chemical reaction or chemisorption to the nanoparticle surface. The description herein can be for the component before it reacts with or chemisorbs with the surface.

The monolayer components can be molecules including organic molecules. The molecules can be polymeric or lower molecular weight. For example, the molecules can have a molecular weight of less than 1,000 or less than 500.

The monolayer components can be adapted to functionalize or bind to the surface by for example chemisorption. Sulfur-containing compounds can be used like mercapto compounds or thiols or disulfides. For example, sulfur bonding to silver or gold metal can be used.

The monolayer component can be represented by A-B—C, wherein A is a functional group adapted to bind or bond to a nanoparticle surface, C is a functional group which can be reactive to bind to an external moiety for further functionalization of the nanoparticle surface, and B is a linking moiety such as a methylene or methylene repeat —CH₂)ₙ— moiety to link together A and C covalently.

For example, A can be a sulfur-containing group such as an —SH group or a disulfide.

C can be for example a hydroxyl group or a methyl group.

B can be for example a methylene linking group such as —[CH₂], where n can be any acceptable number such as for example 1 to 30 or 2 to 30 or 3 to 30 or 4 to 20. B can be linear or branched but generally is linear. B can have for example three to 30 carbons, or four to 20 carbons.

Monolayer components can be used to generate dispersibility in water including use of monolayer components which have hydrophilic, or polar, end groups which are directed to the nanoparticle surface when the nanoparticle is in water. Monolayer components can be used which have some hydrophobicity, but when the water dispersion is desired, the hydrophilic components will be used more than the hydrophobic components.

Particular examples of thiol-containing molecules, or mercapto compounds, that can be utilized for self-assembly on the surface of noble metal nanoparticles either directly or once functionalized can selected and used in amounts to facilitate water solubility. Monothiols can be used. Dithiols can be used. In some cases, some hydrophobic moieties may be used although generally water dispersible moieties are used and the amount of any hydrophobic monolayer component is minimized or eliminated. The number of carbon atoms can be, for example, about 30 or less. Examples include 1-adamantanethiol, 11-amino-1-undecanethiol hydrochloride, biphenyl-4,4′-dithiol, Butyl 3-mercaptopropionate, m-Carborane-1-thiol, m-Carborane-9-thiol, Copper(I) 1-butanethiolate, 4-Cyano-1-butanethiol, S-(4-Cyanobutyl)thioacetate, 1-Decanethiol, 3-(Dimethoxymethylsilyl)-1-propanethiol, 1-Dodecanethiol, tert-Dodecylmercaptan, 2,2′-(Ethylenedioxy)diethanethiol, 2-Ethylhexanethiol, 6-(Ferrocenyl)hexanethiol, 1-Heptanethiol, 1,16-Hexadecanedithiol, 1-Hexadecanethiol, Hexa(ethylene glycol)mono-11-(acetylthio)undecyl ether, 1,6-Hexanedithiol, 1-Hexanethiol, 4-Mercapto-1-butanol, 3-Mercapto-2-butanol, 12-Mercaptododecanoic acid, 16-Mercaptohexadecanoic acid, 16-Mercaptohexadecanoic acid, 6-Mercaptohexanoic acid, 6-Mercapto-1-hexanol, 4-(6-Mercaptohexyloxy)benzyl alcohol, 9-Mercapto-1-nonanol, 3-Mercapto-N-nonylpropionamide, 8-Mercaptooctanoic acid, 15-Mercaptopentadecanoic acid, 1-Mercapto-2-propanol, 3-Mercapto-1-propanol, 3-Mercaptopropionic acid, Mercaptosuccinic acid, 11-Mercaptoundecanoic acid, 11-Mercaptoundecanoic acid, 11-Mercapto-1-undecanol, 11-Mercapto-1-undecanol, (1-Mercaptoundec-11-yl)hexa(ethylene glycol), 11-Mercaptoundecylphosphoric acid, (1-Mercaptoundec-11-yl)tetra(ethylene glycol), 1-Mercaptoundecyl trifluoroacetate, [1-(Methylcarbonylthio)undec-11-yl]tetra(ethylene glycol), Methyl 3-mercaptopropionate, NanoTether BPA-HH, NanoThinks™ 8, NanoThinks™ 18, NanoThinks™ ACID11, NanoThinks™ ACID16, NanoThinks™ ALCO11, NanoThinks™ THIO8,1,9-Nonanedithiol, 1-Nonanethiol, 1-Nonanethiol, 1-Octadecanethiol, 1,8-Octanedithiol, 1-Octanethiol, 1-Pentadecanethiol, 1-Pentanethiol, 1H,1H,2H,2H-Perfluorodecanethiol, 1-Propanethiol, 1-Tetradecanethiol purum, Triethylene glycol mono-11-mercaptoundecyl ether, 1,11-Undecanedithiol and 1-Undecanethiol.

### First Monolayer Component

The first monolayer components can be adapted to provide the monolayer and nanoparticle with binding functionality, including binding to biological moieties and covalent binding and specific binding.

In one embodiment, the nanoparticle is adapted to have on average one first monolayer component per particle.

The first monolayer component can comprise a terminal functional group reactive to biological moieties and in particular peptide and proteins including for example a carboxyl group, whether in acid or base form. In other words, in A-B—C, C can be carboxyl.

The first monolayer component can be a mercapto compound. An example of the first monolayer component is 11-mercaptoundecanoic acid (MUA).

In many cases, the first monolayer component will have a molecular weight less than the molecular weight of the second monolayer component.

### Second Monolayer Component

The second monolayer component, different from the first, can be adapted to provide nanoparticle stability, particularly in water, and to allow exposure of the first monolayer component.

The second monolayer component can be a mercapto compound. An example for the second monolayer component is 6-mercapto-1-hexanol (MCH).

### Third Component

A third component may be present also which is different from the first and second components, and may be present as a result of the initial synthesis of the nanoparticle, before introduction of the first and second monolayer components. The component can help further stabilize the particle to aggregations, and components known in the art to improve stability can be used, including organic components, whether neutral or ionic.

The third component can be for example a low molecular weight component such as for example citrate or a high molecular weight component such as for example a polymer, including for example a synthetic polymer, including for example a water soluble synthetic polymer, including for example a polar polymer such as for example poly(vinyl pyrrolidone) (PVP). Polymers known in the art which can stabilize particles can be used. Polymers are known in the art. See for example Allcock, Lampe, Contemporary Polymer Chemistry, Prentice-Hall, 1981.

### Amounts of the Monolayer Components

The amounts of the monolayer components on each particle on average can be adapted to provide the nanoparticle, or collection of nanoparticles, with one or more desirable characteristics like stability to aggregation. In particular, the molar ratio of the amount of the first monolayer component to the amount of the second monolayer component can be adapted to provide a stability against aggregation when a collection of the nanoparticles is dispersed in water. In addition, the molar ratio of the amount of the first monolayer component and the amount of the second monolayer component can be adapted to provide sufficient functionalization for further binding of the particle. For example, on average, each particle may have one or two binding moieties from the second monolayer component. More particularly, the molar ratio of the amount of the first monolayer component to the amount of the second monolayer component can be adapted to provide a stability against aggregation of at least 30 days or at least one month when a collection of the nanoparticles is dispersed in water, as measured by size and color.

The amount of the monolayer components can be measured by methods known in the art including for example NMR. A mole ratio can be measured.

For example, the molar ratio of the first and second components can such that there is more of the second component than the first component. The ratio can be for example more than about 1:1 to about 1:20, or more than about 1:1 to 1:15, or more than about 1:1 to 1:10, or more than about 1:1 to about 1:5, or about 1:2 to about 1:20, or about 1:2 to about 1:15, or about 1:3 to about 1:15.

The molar amount of the third component can be larger than the amount of the first or the amount of the second component, or more than the amount of the sum of the first and second components. For example, the molar amount of the third component can be at least 50%, or at least 75%, or at least 90% of the total amount of the three components. Examples of ranges of amounts of the three components (first, second, and third respectively) including about 1:2:10 to about 1:5:50. Or in other words, if the amount of the first component is fixed at 1, the amount of the second component can be about 2 to about 5, and the amount of the third component can be about 10 to about 50.

### Solutions or Dispersions of Nanoparticles

The nanoparticle can function as a collection of nanoparticles dispersed in a solvent or solvent system, or also called a solution.

Statistical averages can be used to characterize the nanoparticles including for example average particle size and standard deviation in particle size. The degree of monodispersity can be measured.

Stable solutions can be prepared.

The dispersant can be water, buffered water, and other systems useful in for example the biochemical arts. Organic solvents can be used in amounts as appropriate. The dispersion is preferably buffered water or pure water, free from organic solvent, for biological applications.

### Stability of the Nanoparticle

Methods known in the art can be used to measure the nanoparticle stability in water or buffer solution. For example, particle size can be measured over time. The spectra including for example color (spectra) of nanoparticles can be measured over time.

The change in parameter can be less than 10%, or less than 5% or less than 1%, over 15 days, less than 10%, or less than 5% or less than 1%, over 30 days, less than 10%, or less than 5% or less than 1%, over 60 days, and less than 10%, or less than 5% or less than 1%, over 90 days.

The particles can be stable, which resist aggregation and photodecomposition.

### Method of Making Nanoparticles

Methods known in the art can be used to make the nanoparticles. A multi-stage process can be used. For example, in a first stage, a nanoparticle can be prepared. In a second stage, the nanoparticle can be further functionalized. In a third stage, the nanoparticle can be even further functionalized with a moiety adapted to bind to an analyte.

In one approach, a metal component can be provided, and the metal can be subjected to a reduction reaction with use of a reducing agent. For example, a metal cation can be provided, or a metal in an anion can be provided. For example, a silver salt can be used such as silver perchlorate or silver nitrate. A plurality of reducing reagents can be used such as for example a mixture of a citrate salt (e.g., sodium citrate) and a borohydride salt (e.g., sodium borohydride).

Reaction can be carried out in water or aqueous media.

The combination of synthetic variables such as reaction parameters like time, temperature, mixing and stirring speed, and concentrations of reagents, particularly reaction time, can be an important parameter to providing stable nanoparticles and controlling particle size. For example, longer reaction times can favor larger particles and shorter reaction times can favor smaller particles. Stabilizers can be used to control nanoparticle growth.

In one embodiment, an initial surface layer can be disposed on the nanoparticle surface, and then exchange reactions can be carried out to replace at least some of the initial component with one or more new components. The replacement reaction can be controlled so that the nanoparticles do not aggregate during the reaction and remain stable over weeks and months of time.

Principles of SN1 and SN2 substitution can be used in functionalizing the nanoparticle surface and ligand exchange and maintaining stability of the nanoparticle. See, for example, Smith, March, March's Advanced Organic Chemistry, Wiley, 2007, including description for SN2 and SN1 mechanisms, and leaving groups and unimolecular and bimolecular reactions. The SN2 pathway may encourage nanoparticle stability. See for example working example below.

In making nanoparticles, centrifugation may be an important step. See for example working examples below. A proper centrifuge speed can be selected, and this can be particularly important for smaller nanoparticles which have for example nanoparticle size of less than about 10 nm or less than about 5 nm. For example, if the centrifuge speed is too high, the centrifugation may produce aggregation. If the centrifuge speed is too low, the nanoparticles may not settle.

Nanoparticles can be made without use of lithography such as nanosphere lithography (NSL), but rather solution methods can be used apart from lithography.

### Further Functionalization of the Nanoparticle

In addition to functionalizing the nanoparticle with the first monolayer component and the second monolayer component, the nanoparticle can be further functionalized or coupled to allow the nanoparticle to bind to an analyte. One or more of the monolayer components on the nanoparticle can be further functionalized. For example, the nanoparticle can be functionalized with at least one biological moiety. Biological moieties are known in the art. See for example Bohinski, Modern Concepts in Biochemistry, 4^{th} Ed., 1983, including moieties based on amino acids, peptides, proteins, enzymes, nucleotides, nucleic acids, carbohydrates, lipids, biomembrane materials, molecules and chemicals associated with the immune and natural defense systems, cellular compartment materials and molecules, and the like. See, for example, The Molecular Biology of the Cell, 2^{nd} Ed., 1989; ImmunoBiology, 5^{th} Ed., 2001; and Clinical Immunology, Principles and Practice, Vol. I, II, 1996. Covalent functionalization can be carried out.

The biological moiety which functionalizes the nanoparticle can be capable of specific binding including for example antigen-antibody binding, or receptor-ligand binding, or enzyme-substrate binding. See, for example, Fersht, Enzyme Structure and Mechanism, 2^{nd} Ed., 1977.

Conditions can be adapted so that on average one nanoparticle is associated with one biological moiety.

Coupling methods known in biochemistry can be used.

### Analyte

Analytes are generally known in the art and can be a target for analysis, imaging, or detection for the functionalized nanoparticles. A wide variety of analytes can be used including for example biological analytes, organic analytes, and inorganic analytes. The analyte can be in solution or on a surface. Analytes can be present in very low amounts and concentrations, and single molecules of analyte can be detected in some cases. In particular, the analyte can be a proteinaceous materials including proteins and peptides. The analyte can be adapted for the applications as described more below.

Peptides are polymers of amino acids ordered in a highly specific sequence through covalent chemical bonds known as amide or peptide bonds. Peptides are generally considered to be short sequences of amino acids, however many examples of long peptides are known to exist and have important biological functions.

Proteins are found in various forms and can be polypeptides or groups of multiple polypeptide subunits. Proteins range in size but are generally considered be larger than peptides.

Antibodies are examples of proteins that are produced by the immune system of vertebrates and function to recognize a specific site, known as an epitope, on a target molecule that is also known as an antigen. Antibodies that recognize multiple epitopes of their antigen are know as polyclonal antibodies, while antibodies that recognize and bind to only one antigen are know as monoclonal antibodies. Polyclonal antibodies can be produced by injecting a desired antigen into an animal, initiating an immune response, then subsequently purifying the antibody from the blood of the animal. Monoclonal antibodies can be generated by fusing myeloma cells with spleen cells from an animal that has been immunized with the desired antigen. Due to their high degree of specificity, monoclonal antibodies are commonly utilized to detect target molecules in diverse areas that include research and diagnostics tools, biosensors and environmental.

Monoclonal antibodies produced using mouse cells may, however, elicit an immune response in a non-mouse animal. To generate monoclonal antibodies that can be administered to non-mouse animals, recombinant DNA technology is utilized to merge the DNA that encodes the binding portion of a monoclonal mouse antibody with the antibody producing DNA of the desired vertebrate animal, such as a human. One then uses an in vitro cell culture system to express this merged DNA and produce the chimeric antibody. If the antibody comprises half mouse DNA and half human DNA the antibody is said to be humanized. In this way, monoclonal antibodies can be administered to humans for therapeutic purposes without the host immune system recognizing the antibody and generating an undesired immune response.

Antibodies can be further modified through the process of glycosylation to improve the antibody-dependent cell mediated cytotoxicity (ADCC). Improving the ADCC results in a monoclonal antibody that, upon binding to a desired target cell such as a cancer cell, will destroy the target cell.

### Binding of Nanoparticles to Analytes

A further composition can be formed when the nanoparticle binds to an analyte. The binding process can be measured by methods known in the art and described below. For example, binding constants and kinetics and rates can be measured. Specific binding is often desirable. Examples of specific binding are known in the art including, for example, streptavidin-biotin and antibody-antigen.

### Applications and Uses

Many biological applications can be carried out including for example sensors, diagnostics, pharmacological, and drug delivery. Both in vivo and in vitro studies can be carried out. In particular, applications can be carried out wherein trace amounts of analytes can be detected. Also, quantitative detection can be carried out. Other applications can be found in for example New Frontiers in Ultrasensitive Bioanalysis: Advanced Analytical Chemistry Applications in Nanobiotechnology, Single Molecule Detection, and Single Cell Analysis, X. Xu, Ed., Wiley, 2007, and also Bourne, A Consumer's Guide to MEMS & Nanotechnology, Bourne Research, 2007, including Chapter 11 on health care applications and diagnostic tools. See also, Nature Biotechnology, 27, 7, Jul. 2008 (729-732). Cancer research applications are noted in NCI Alliance for Nanotechnology in Cancer, Monthly Feature, May 2006.

In particular, single molecule detection (SMD) can be carried out.

Single living cells can be studied. Cells can be prokaryotic or eukaryotic. Cell receptors can be studied. Genetic engineering methods can be used to prepare cells and cell surfaces for analysis.

Single nanoparticles can be detected and imaged.

Cell culturing and assay methods known in the art can be used.

In particular, a use is as ultrasensitive biosensor for detecting, sensing, and imaging of single protein molecules on single living cells.

In particular, a use is in vivo imaging for diagnosis and therapy and drug delivery, as well as fundamental research. An example is using single nanoparticle biosensors to characterize anti-cancer vaccines at single molecule level for rational design of anticancer vaccines for effective therapy.

In particular, a use is assay and biosensor that exhibits extraordinary high sensitivity, selectivity, and wide dynamic range of 0-200 ng/mL, which is well suited for study of biological function in single biomarker molecules such as for example TNF-alpha and development of effective earlier disease diagnosis and therapy.

Applications are preferred wherein fluorescence spectroscopy is not used to detect binding, and the system is free of fluorophore noise and photobleaching, and the need of expensive excitation source and detection schemes related to fluorescence imaging and detection.

In addition, kits can be prepared which can include one or more components or reagents and also instructions for use of the components or reagents. The kits can be used in, for example, assaying.

### Sensing on Surface Applications

In one particular type of application, surfaces are examined including solid surfaces, substrate surfaces, and biological surfaces including for example cellular surfaces such as cell membrane surfaces. Receptor mapping can be carried out. Individual receptor molecules can be detected.

### Sensing in Solution Applications

In another type of particular application, the analyte is detected in solution in particular at low concentrations.

For example, analyte can be detected at levels less than about 10⁻⁷ M, less than about 10⁻¹² M, or less than about 10⁻¹⁵ M. Single molecule detection can be achieved including, for example, detection at concentrations of about 100 nM or less.

Additional description is provided by way of non-limiting working examples as follows.

### WORKING EXAMPLES

### Working Examples Part I

### A. “Experimental Section: Reagent Supplies to Data Analysis and Statistics”

### Reagents and Supplies.

Sodium citrate dihydrate (≧9%), AgClO4 (≧9.9%), NaBH4 (≧8%), 11-mercaptoundecanoic acid (MUA; ≧5%), 6-mercapto-1-hexanol (MCH; ≧97%), trypan blue (0.4%), immunoglobulin G from rabbit serum (IgG; ≧5%), protein A (PrA) from Staphylococcus aureus (≧95%), collagen from calf skin, bovine serum albumin (BSA), 30-kDa poly(ethylene glycol) (PEG), and ethanol (≧99.8%) were purchased from Sigma-Aldrich. 1-Ethyl-3-(3-dimethylaminopropyl)-carbodiimide hydrochloride (EDC; ≧9%) and N-hydroxysulfosuccinimide (Sulfo-NHS; ≧8.5%) were purchased from Pierce. Dulbecco's modified Eagle's medium (DMEM) 1× medium, 100× penicillin-streptomycin-glutamine solution (P—S-G), fetal bovine serum (FBS), Hanks' balanced salt solution (HBSS, without Ca²⁺, Mg²⁺ and phenol red; pH 7.4), and mouse fibroblast cells (L292) were purchased from ATCC. The T-ZZ was constructed by fusing the C-terminus of transmembrane domain (T) of diphtheria toxin (a 58-kDa soluble protein) with ZZ using protein engineering techniques.^{A28-A30} The T domain (22 kDa) isolated from the rest of diphtheria toxin^{A34} is devoid of toxicity. All solutions, including 10 mM phosphate buffer (PB) (pH 7.4), 10 mM phosphate-buffered saline (PBS) (pH 7.4), and 10 mM citrate phosphate buffer saline (pH 4.8), were prepared using 18-MΩ Nanopure water (Barnstead).

Synthesis and Characterization of Single-Nanoparticle Biosensors.

Ag Nanoparticles. The sodium citrate (3 mM) and NaBH₄ (10 mM) in Nanopure water (495 mL) was prepared freshly and stirred constantly at 0° C. As AgClO₄ (5 mL, 10 mM) was added into the solution, the color of the solution changed from colorless to yellow. After stirring for 4 h at room temperature, the solution was filtered using 0.2-μm membrane filters. The diameter of Ag nanoparticles is 11.6±3.5 nm as characterized by HRTEM (FEI Tecnai G2 F30 FEG) and dynamic light scattering (DLS) (Nicomp 380ZLS particle sizing system). AgMMUA Nanoparticles. The mixture (5 mL) of MUA (10 mM) and MCH (90 mM) in ethanol was added into the freshly prepared Ag nanoparticle solution (500 mL) and stirred for 48 h. The AgMMUA nanoparticles were harvested and washed twice with Nanopure water to remove excess thiol by centrifugation (12,000 rpm, 4° C., ˜150 min). The AgMMUA nanoparticles were characterized using UV-vis spectroscopy (Hitachi U3310), NMR (400 MHz, Bruker), DLS, and SNOMS. NMR samples were prepared by rinsing AgMMUA nanoparticles with Nanopure water three times using centrifugation, dialyzing using a 30-kDa cutoff member, drying using lyophilization (VirTis), and dissolving 50 mg of AgMMUA nanoparticles in 1.0 mL of D₂O. AgMMUA-IgG Nanoparticles. The EDC (26.4 nmol) and sulfo-NHS (35.2 μmole) were added to AgMMUA aqueous solution (3 mL, 80 nM), to generate AgMMUA-s-NHS. After stirring at room temperature for 40 min, AgMMUA-s-NHS was desalted using a Centriprep YM-30 (Millipore) by centrifugation at 1500 relative centrifuge force to remove excess EDC and sulfo-NHS and then redissolved in PBS buffer. PEG (0.05% w/v) was added to block any possible nonspecific binding sites on the AgMMUA nanoparticles and to prevent nonspecific adsorption of IgG onto the surface during the following linking reaction. Then, IgG was added to AgMMUA-s-NHS in PBS buffer to a mole ratio of IgG to AgMMUA of 0.97. The solution was mixed using a rotary shaker at room temperature for 2 h and then at 4° C. for 12 h. The final product (AgMMUA-IgG nanoparticles) was washed with PBS buffer to remove excess IgG using centrifugation (10,500 rpm, 4° C., ˜90 min). The pellet was resuspended and stored in PBS buffer containing 0.05% w/v PEG at 4° C. for future use. PrA in PBS buffer solution was added into the AgMMUAIgG nanoparticle solution in a quartz cuvette and carefully mixed by pipet. The UV-vis spectra of the mixture was measured immediately and followed for 24 h to monitor the binding kinetics of AgMMUA-IgG with PrA. The spectra were taken every 5 min at room temperature for the first 2 h and then every 2.5 h for 24 h. The solution was stored at 4° C. to prevent denaturation of the proteins between spectroscopy measurements.

### Cell Culture and Viability Assay.

The fibroblast cells (L929) were first grown in a culture flask using the medium DMEM 1× with 1% P—S-G and 10% FBS. When the cells reached 80% confluence, they were transferred from the flask and cultured directly on sterile coverslips, which were precoated with 1 mg/mL collagen in 0.1 M acetic acid solution, rinsed, and dried. The cells (0.1 mL, 1×10⁶ cells/mL) were carefully added onto each coverslip to ensure uniform coverage and allowed to adhere onto the coverslips. The cell medium was then added to cover the coverslips in Petri dishes, which were placed in a CO₂ incubator (5% CO₂, 100% humidity, 37° C.) overnight. The inventors also investigated cell viability through the entire experiment using a Trypan blue cell viability assay. The cells incubated with Trypan blue (0.4%) were imaged within 5-min incubation time at the single-cell resolution using bright-field microscopy, and stained (dead) and unstained (viable) cells were counted. More than 300 cells in total were investigated for each data point.

### Imaging and Characterization of Single Receptor Molecules on Single Living Cells.

The living cells (105) cultured on each coverslip were thoroughly rinsed with HBSS buffer (pH 7.4) and PBS-citrate buffer (pH 4.8) to remove the medium and incubated with 40 μL of 0.10 or 1.0 μM T-ZZ in PBS-citrate buffer at 4° C. for 45 min to anchor T-ZZ on the cell surface. The cells were first rinsed with PBS-citrate buffer, then with HBSS buffer to thoroughly remove unbound T-ZZ, finally incubated with 1 μM BSA protein in HBSS buffer for 15 min, aiming to block any possible nonspecific interaction of the cell membrane with protein (AgMMUA-IgG), and rinsed thoroughly with HBSS buffer to remove unbound BSA. The cells on the coverslips were placed onto the slides, incubated with AgMMUA-IgG nanoparticles (1.39, 2.78, 5.56, and 11.12 nM) in PBS buffer (pH 7.4) in a well-sealed chamber created on the slide, and imaged using our SNOMS at given reaction times of 0-180 min (0, 20, 40, 60, 80, 100, 120, 150, and 180 min). Water droplets were scattered around the slide to maintain humidity, preventing possible evaporation of buffer solution inside the chamber. The nanoparticle concentrations were calculated as described in our previous study and presented in Supporting Information (SI).^{A16,35}

SNOMS has been well described in our previous studies for real-time imaging and spectroscopic characterization of single nanoparticles on single living cells and for single-molecule detection.^{A13,15,16,23,25,27,36} The detectors (5 MHz Micromax CCD camera and color digital camera) were used for imaging of single nanoparticles (Ag, AgMMUA, AgMMUA-IgG) and single living cells, and EMCCD or LN back-illuminated CCD camera coupled with a SpectraPro-150 (Roper Scientific) was used for measuring localized surface plasmon resonance spectra (LSPRS) of single nanoparticles.

Three blank control experiments were carried out simultaneously for each experiment: cells anchored with two surface coverages of T-ZZ prepared exactly the same as described above were incubated with 11.12 nM AgMMUA nanoparticles (not yet conjugated with IgG) and imaged for 0-180 min; cells without attaching of T-ZZ were incubated with 11.12 nM AgMMUA-IgG nanoparticles and imaged exactly the same way.

### Data Analysis and Statistics.

At each given reaction time, the cells at 15-20 representative locations on the coverslips were imaged and at least 40 cells were studied for each measurement. Each experiment was repeated at least three times. Thus, 120 cells per data point were imaged to gain sufficient statistics to represent the bulk cells at single-cell resolution.

### Synthesis and Characterization of Single-Nanoparticle Biosensors.

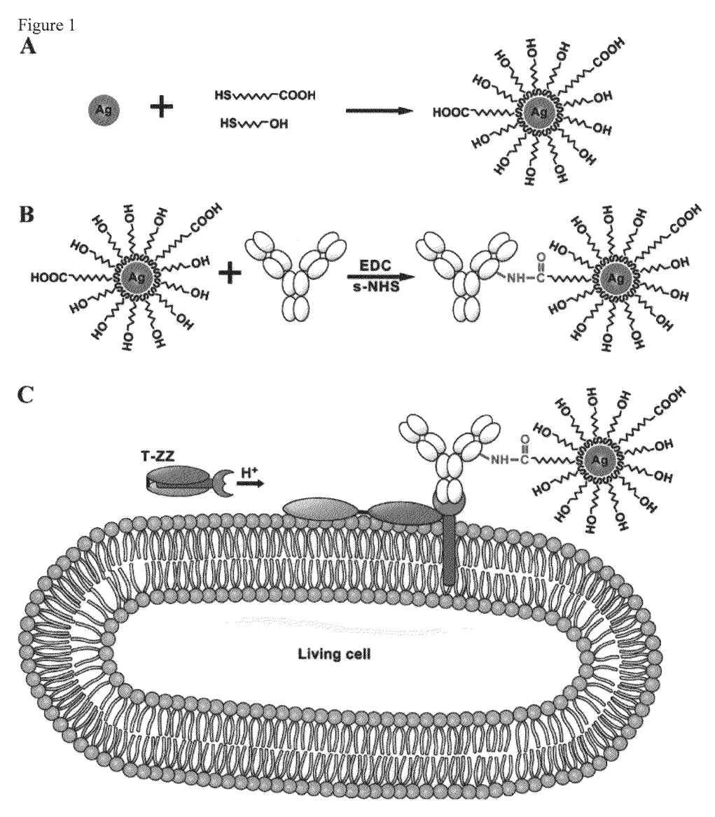

In accordance with the present invention, the inventors synthesized nearly monodispersed (11.6±3.5 nm in diameter) Ag nanoparticles as characterized by HRTEM (shown in FIG. 1S of SI) by carefully selecting experimental conditions for reduction of AgClO₄ with sodium citrate and NaBH₄. Then, the inventors functionalized these Ag nanoparticles with a mixed monolayer of MUA and MCH using the interaction of the thiol group (SH) with the Ag nanoparticle surface to prepare AgMMUA (FIG. 1). Note that the Ag nanoparticles were stabilized in solution by electrostatic repulsion of the surface-adsorbed charged citrate layer.³⁷⁻³⁹ If only MUA was used to replace the adsorbed citrate layer, the replacement reaction would follow a unimolecular nucleophilic substitution (SN1) mechanism, because the citrate is a very good leaving group, and the charge repulsion and steric hindrance of MUA makes an SN2 mechanism nearly impossible to occur.^{A37-39} Thus, the presence of the MUA would accelerate the dissolving of the charged citrate layer, leading to the aggregation of nanoparticles, which is the major problem in synthesis of ω-mercaptoalkanoic acid-modified Ag nanoparticles. To solve this problem, the inventors used a mixture of MUA and MCH with a mole ratio of 1:9. The presence of an excess of noncharged short-chain MCH allows the substitution reaction more favorable to a SN2 mechanism, which prevents the aggregation of the Ag nanoparticles. The hydroxyl group on the end of MCH makes the functionalized Ag nanoparticles more hydrophilic. Since MCH is much shorter than MUA, it will not block the carboxyl group of the MUA for further linking with IgG. The short chain of MCH is more rigid than MUA molecules; therefore, MUA surrounded by the rigid short-chain thiols of MCH is more likely to stand straight on the nanoparticle surface (FIG. 1A) rather than lay flat on the surface of the Ag nanoparticles, which reduces steric hindrance and allows the MUA to link with IgG more effectively. With this approach, the inventors successfully avoided aggregation of nanoparticles and prepared AgMMUA nanoparticles that are stable (size and color of single nanoparticles remain unchanged) in aqueous solution (Nanopure water and buffer solution) for months. Although a reaction scheme had been used to functionalize the Au surface^{A40} and nanoparticles immobilized on the surface,^{A18} it has not yet been achieved for Ag nanoparticles that freely diffuse in the solution. The inventors found that the ratio of MUA to MCH and reaction time were important to preventing aggregation of nanoparticles.

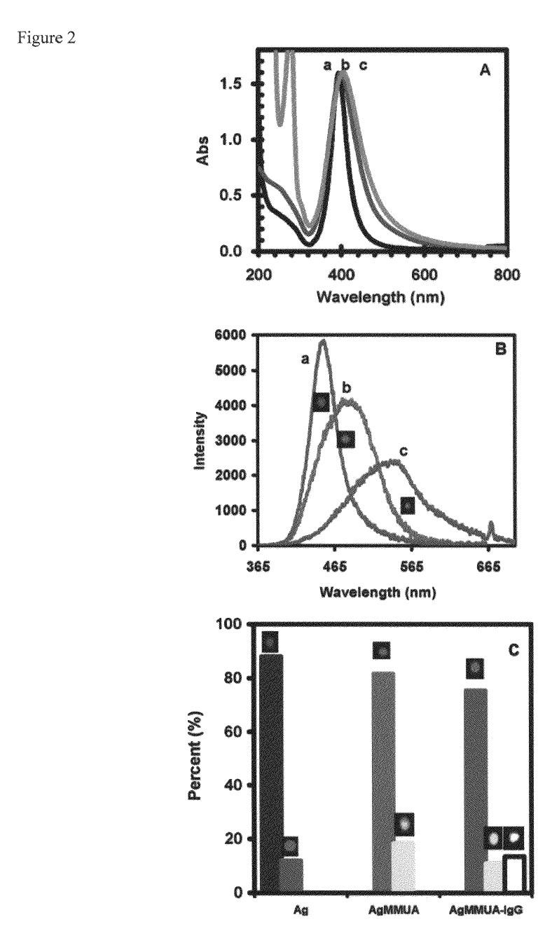

NMR spectra show that MUA, MCH, and citrate are attached on the surface of Ag nanoparticles with a mole ratio of 1:3:30 (FIG. 2S in SI). The large and broad chemical shift at 2.5 ppm is from the —CH2- next to the thiol of both MUA and MCH,^{A41} and citrate adsorbed on the Ag nanoparticle surface.^{A42} Peak broadenings are observed at all chemical shifts of the NMR spectra, indicating that the MUA and MCH are on the surface of Ag nanoparticles, because nanoparticles attached to MUA and MCH slow down the rotational motion of these molecules.^{A43-45} Furthermore, the gradient of packing density along the functional groups may also contribute to the peak broadening.

Finally, the inventors linked the carboxyl group of AgMMUA with the amine group of IgG via a peptide bond to prepare single nanoparticle biosensors (AgMMUA-IgG), as illustrated in FIGS. 1A and B. By controlling the mole ratio of IgG to AgMMUA nanoparticles at less than 1 (0.97) during the conjugation reaction, the statistical average conjugation ratio is limited to one IgG molecule/nanoparticle. The nanoparticle biosensors prepared using covalent conjugation have several advantages over those prepared using electrostatic interaction. They are more stable and the conjugation ratio remains unchanged in buffer solution of various ionic strength and pH, allowing quantitative analysis of individual protein molecules on single living cell surface.

The representative UV-vis absorption spectrum of bulk Ag, AgMMUA, and AgMMUA-IgG nanoparticle solution shows peak wavelengths at 393, 400, and 406 nm, respectively, illustrating a red shift as the surface of Ag nanoparticles is modified with the functional groups (FIG. 2A). The LSPRS of representative single Ag nanoparticles from these three solutions exhibits peak wavelengths of 450, 482, and 545 nm, respectively, showing a longer wavelength and lower intensity as the surface of Ag nanoparticles is functionalized with the mixed monolayer of MUA and MCH and linked with IgG (FIG. 2B). The histograms of the different colors (LSPRS) of individual nanoparticles of the three solutions in FIG. 2C shows that Ag nanoparticles in Nanopure water contains blue (88%) and green (12%) nanoparticles; AgMMUA nanoparticles in Nanopure water includes the light green (82%) and yellow green (19%) nanoparticles; and AgMMUA-IgG nanoparticles in PBS buffer has dark green (75%), yellow (11%), and white (14%) nanoparticles.

As described by Mie theory^{A46} and demonstrated by previous studies,^{A13,15,16} the LSPRS and scattering intensity of single Ag nanoparticles highly depend on its size, shape, surface properties, and surrounding environments. The color distribution of individual nanoparticles further illustrates that the functional groups on the surface of Ag nanoparticles lead to changes of the optical properties of individual nanoparticles and the red shift of their LSPRS. The white color of nanoparticles observed in the AgMMUA-IgG solution is most likely attributable to the crosslinking of nanoparticles, leading to larger nanoparticles, which can be removed via centrifugation.

### Characterization of Biological Activity of Single-Nanoparticle Biosensors.

The primary challenge of preparing effective nanoparticle biosensors, especially by covalent conjugation of protein molecules (IgG, ligand) with nanoparticles, is to retain the biological activities of those biomolecules attached on the surface of nanoparticles. To characterize the biological activity of AgMMUA-IgG, the inventors measured their binding affinity with PrA in buffer solution and compared their binding constant with that of IgG-PrA in solution.

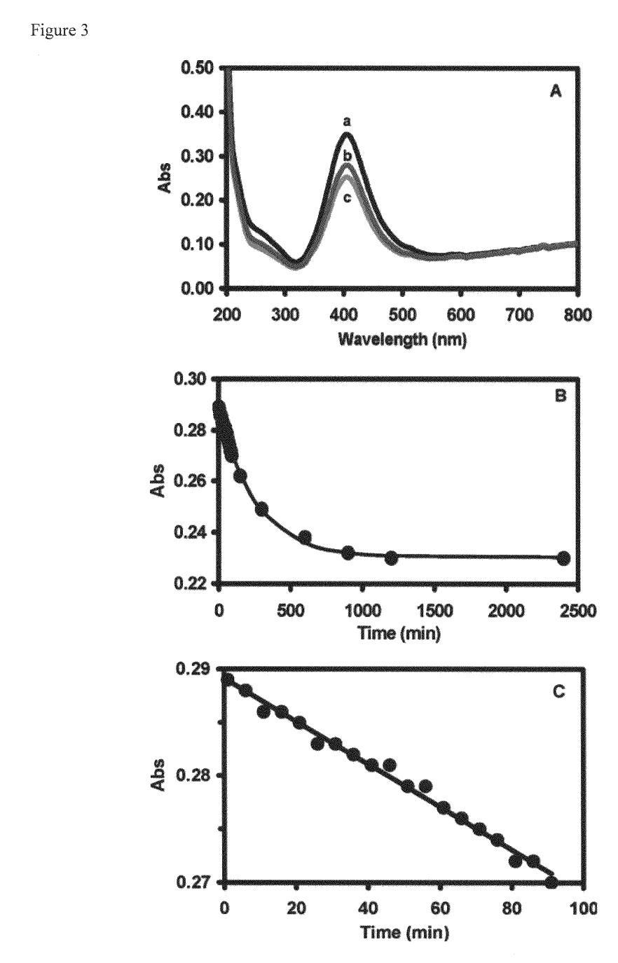

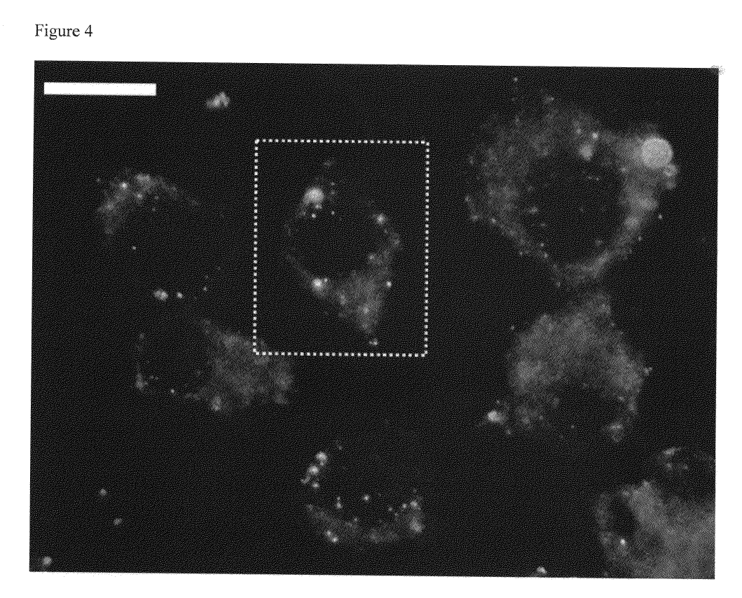

The results in FIG. 3 illustrate that the absorbance of AgMMUA-IgG nanoparticles decreases and reaches a constant value, indicating that AgMMUA-IgG nanoparticles bind with PrA and reach binding equilibrium. It is well-known that PrA (a receptor) displays 3-4 binding sites for the Fc portion of rabbit IgG,^{A9} which allows one PrA molecule to bind with multiple AgMMUA-IgG nanoparticles, leading to the precipitation of bound IgG-PrA.^{A49} The peak wavelength of the spectra remains constant over time (FIG. 3A), suggesting that AgMMUA-IgGPrA nanoparticles did not contribute significantly to the absorbance and indicating that cross-linked AgMMUA-IgG-PrA nanoparticles precipitate from solution. Otherwise, the inventors would observe a red shift of peak wavelength if the cross-linked larger AgMMUA-IgG-PrA nanoparticles remained suspended in the solution. The extinction coefficient (molar absorptivity, ∈₁) of AgMMUA-IgG calculated from the absorption spectra using the Beer-Lambert law is 2.6×10⁸ M⁻¹ cm⁻¹.

Plot of peak absorbance subtracted from baseline versus time in FIGS. 3B and C demonstrates a nearly first-order reaction as illustrated below:

AgMMUA-IgG+PrA [symbol] AgMMUA-IgG-PrA [1]

Since the PrA molar concentration is 100 times higher than that of AgMMUA-IgG, the inventors assume the PrA concentration remains constant over the entire reaction. Thus, a second-order (or multiple-order) reaction can be treated as a first-order reaction. Using the change of AgMMUA-IgG absorbance resulting from its binding with PrA, the inventors calculate the amount of AgMMUA-IgG nanoparticles (0.14 nM) that bound with PrA and the amount of AgMMUA-IgG-PrA (0.14 nM) that is generated, showing equilibrium concentrations of AgMMUA-IgG (0.96 nM), PrA (99.86 nM), and AgMMUA-IgG-PrA (0.14 nM) and the equilibrium binding constant (affinity, K_{b}) as 1.5×10⁶ M⁻¹.

In accordance with the present embodiments, the inventors have further determined the binding association rate constant (kₐ=1.2×10² M⁻¹ s⁻¹) and dissociation rate constant (k_{d}=5.4×10⁻⁵ s⁻¹) using the slope of FIG. 3C and by matching simulation data with the experimental data in FIG. 3B, respectively. The detailed calculation is shown in SI. Using this approach, the inventors can calculate the binding affinity constant (K_{b}) kₐ/k_{d}=2.2×10⁶ M⁻¹), which agrees well with the K_{b} directly calculated using equilibrium concentrations. The experiments were repeated three times, and the average of K_{b}, (1.9±0.1)×10⁶ M⁻¹, determined by both methods, agrees well with those observed in solution (3×10⁶ M⁻¹) and reported in the literature.^{A50} Thus, a primary biological activity of IgG conjugated with nanoparticles is preserved, allowing AgMMUA-IgG nanoparticles to be used to image and characterize individual receptor molecules on single living cells.

### Imaging and Sensing of Individual Receptor Molecules on Single Living Cells.

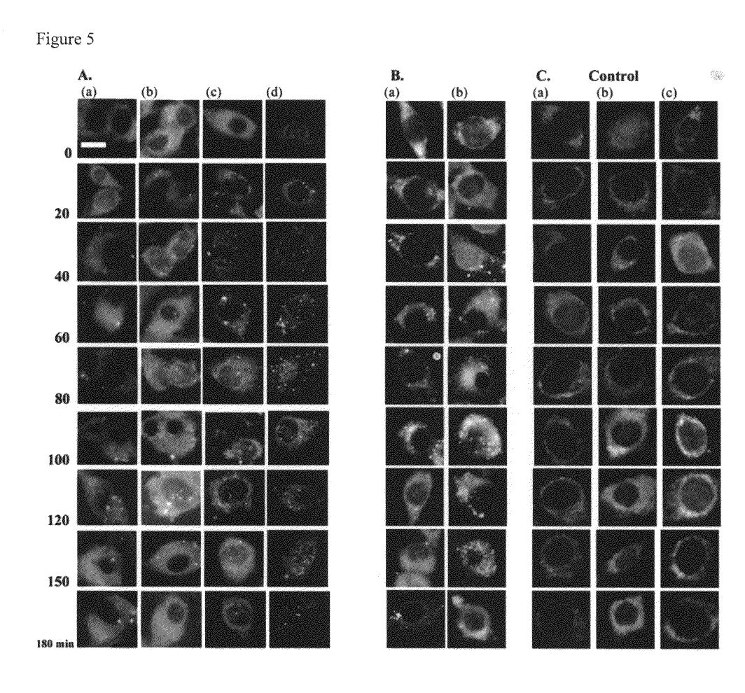

In accordance with the present inventions, the inventors have anchored the T-ZZ onto the membrane of living cells to create the desired low coverage of individual ZZ molecules (0.21-0.37 molecules/μm²) on living cells,^{A28-30} which were detected by single AgMMUA-IgG nanoparticles and imaged in real time using dark-field single nanoparticle optical microscopy and spectroscopy (SNOMS) (FIG. 4). Since the mole ratio of IgG to AgMMUA nanoparticles is 0.97, a single IgG molecule is statistically conjugated with a single AgMMUA nanoparticle. Although multiple IgG molecules may be present in a single AgMMUA-IgG nanoparticle, the low coverage of T-ZZ molecules on the surface of single living cells, and the low concentration of AgMMUA-IgG nanoparticles, greatly reduce the possibility of having more than one T-ZZ molecule bound with a single AgMMUA-IgG nanoparticle, because, at such low coverage (0.21-0.37 molecules/μm²), it is extremely unlikely to have two neighboring T-ZZ molecules within the proximity of a single AgMMUA-IgG nanoparticle (˜15 nm in diameter). If two T-ZZ molecules were located within a cross-sectional area of a single nanoparticle (2.3×10⁴ μm²), the coverage of T-ZZ on a single living cell surface would be 8.7×10⁴ molecule/μm². For those nanoparticles that are not conjugated with IgG, they will not bind with T-ZZ, which will not be detected on cell surface. Thus, no washing step is needed for this assay, and the possible small variation of conjugation ratio of nanoparticles with IgG will not affect SMD of individual receptors on single living cells.

Thus, by controlling a low conjugation ratio of IgG with AgMMUA nanoparticles and low coverage of T-ZZ on the living cell surface, the inventors ensure that a SMD scheme is appropriately applied for the reaction described below:

AgMMUA-IgG+Cell-T-ZZ [symbol] AgMMUA-IgGZZ-T-Cell [2]

Fibroblast cells are highly adhesive cells and were directly cultured on coverslips. Thus, the cells would not move during the experiments. Diffusion of the attached T-ZZ molecules is confined within the cell membrane, which is orders of magnitudes slower than the Brownian motion of single nanoparticles in solution.^{A16,17,51} Therefore, once AgMMUA-IgG nanoparticles bind with T-ZZ on the cell surface, the diffusion of the nanoparticles is negligible in comparison with the free Brownian motion of individual nanoparticles in the solution, showing that the bound AgMMUA-IgG nanoparticles become bright spots shrunk to the resolution limit of the CCD with a 2-pixel diameter. This unique feature allows us to avoid the washing step and directly image the number of individual T-ZZ molecules bound with AgMMUAIgG nanoparticles on the surface of cells. The inventors also acquired sequence images of living cells incubated with AgMMUA-IgG over time and did not observe significant diffusion of T-ZZ bound with AgMMUA-IgG on living cells. The inventors also used the scattering intensity of individual nanoparticles to determine whether the clusters of T-ZZ (more than a single nanoparticle) were present within the spatial resolution of the CCD camera, showing no evidence of aggregation of T-ZZ.

Interestingly, the inventors observed a red shift of the LSPRS of AgMMUA-IgG nanoparticles as they bound with T-ZZ on living cells, showing more orange and red nanoparticles (FIGS. 4 and 5), which may be attributable to the change of the surface properties and dielectric constant of embedded medium of nanoparticles. This interesting phenomenon offers an additional feature, allowing us to distinguish bound and unbound AgMMUA-IgG nanoparticles.

To measure their binding affinity and determine the dependence of binding kinetics on concentration of AgMMUA-IgG nanoparticles, the inventors, in accordance with the present invention, prepared one surface coverage of T-ZZ molecules (0.37 molecules/μm²) on living cells and used four concentrations (1.39, 2.78, 5.56, and 11.12 nM) of AgMMUAgG nanoparticles (Tables 1 and 2 in SI). Although the binding dissociation constant (K_{D}) of IgG with T-ZZ on the living cells has not yet been reported, the K_{D} of T-ZZ with IgG on phospholipids (not on living cell membrane) was estimated as 43 nM by previous bulk measurement.^{A28,52} Thus, the selected concentrations of AgMMUA-IgG nanoparticles mimic the ligand (IgG) concentrations well below its K_{D}.

Using the CCD microarray, the inventors, in accordance with the present invention, were able to image the distribution and binding reaction of T-ZZ molecules on several living cells simultaneously (FIG. 4). Representative cells selected from the full-frame images similar to those in FIG. 4 are illustrated in FIG. 5A, showing the concentration and time dependence of AgMMUA-IgG nanoparticles binding with T-ZZ on living cells. At the same concentration of AgMMUA-IgG, more AgMMUA-IgG nanoparticles bind with T-ZZ molecules on living cells as incubation time increases from 0 to 180 min (the vertical column of FIG. 5A), demonstrating time dependence and realtime monitoring of the binding reaction on living cells. At a given incubation time, as AgMMUA-IgG concentration increases, more AgMMUA-IgG nanoparticles bind with T-ZZ molecules on the cells (the horizontal row of FIG. 5A), showing concentration dependence.

Using the same approach, the inventors, in accordance with the present invention, investigated the dependence of binding kinetics on the coverage of T-ZZ on living cells. The inventors used a single concentration of AgMMUA-IgG nanoparticles (2.78 nM) to detect individual T-ZZ molecules on living cells, which were present with two different coverages of T-ZZ (0.21 and 0.37 molecules/μm²). The inventors observed more AgMMUA-IgG nanoparticles bound with T-ZZ on living cells as incubation time increased (vertical column of FIG. 5B). At the given incubation time, more AgMMUA-IgG nanoparticles bound with T-ZZ on living cells with the higher coverage of T-ZZ (horizontal row of FIG. 5B), showing the dependence of binding reaction rate on the coverage of T-ZZ on cell surface.

To determine possible nonspecific interactions of AgMMUAIgG nanoparticles with living cell membrane, three blank control experiments were performed, by incubating 11.12 nM AgMMUA (not yet conjugated with IgG) with living cells attached with T-ZZ prepared as described in FIGS. 5A and B, and by incubating 11.12 nM AgMMUA-IgG nanoparticles (the highest concentration used in FIG. 5A) with living cells that were not attached with T-ZZ molecules, showing no significant number of bound AgMMUAIgG nanoparticles on living cells (FIG. 5C).

The viability of the cells during each experiment was monitored using a Trypan Blue cell viability assay, showing more than 90% of cells remained alive for the entire experimental duration from treatment with T-ZZ at pH 4.8 for 45 min, rinsing with the buffer at pH 7.4, and imaging in the microchannel for 3 h.

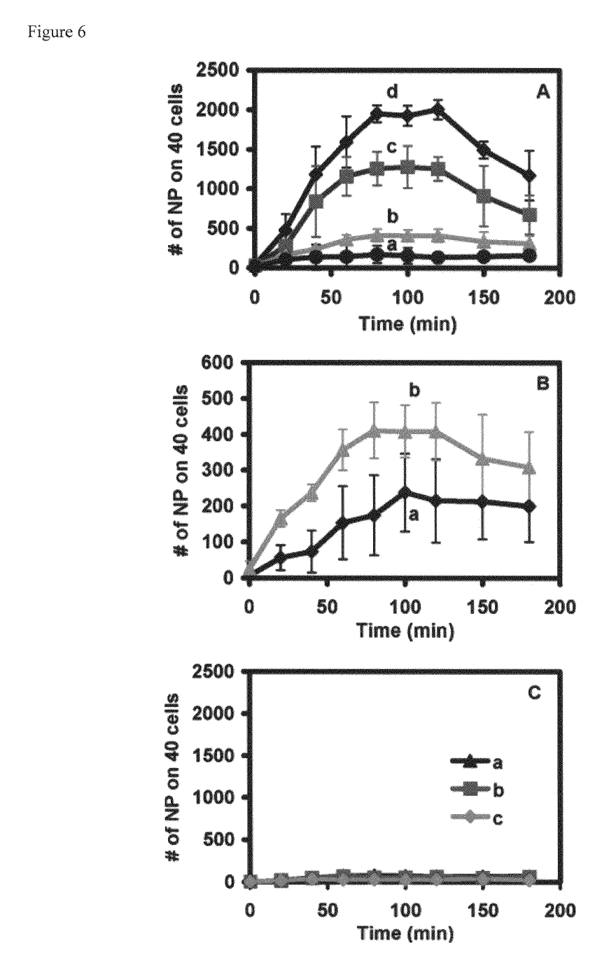

Plots of the number of bound AgMMUA-IgG nanoparticles with T-ZZ on living cells versus reaction time for the study of dependence of AgMMUA-IgG concentration (FIG. 5A), T-ZZ coverage on cell surface (FIG. 5B), and blank control experiments (FIG. 5C) are presented in FIG. 6A-C, respectively. The results further illustrate that binding kinetics depend on ligand concentrations (AgMMUA-IgG) and the coverage of model receptors (T-ZZ) on the cell surface. The control experiments show insignificant nonspecific interaction of AgMMUAIgG nanoparticles with cell membrane.

The inventors determined the binding association (k₁) and dissociation (k₋₁) rate constants, and binding affinity constant (K_{B}) at (9.0±2.6)×10³ M⁻¹ s-1, (3.0±0.4)×10⁻⁴ s⁻¹, and (3.0±0.6)×10⁷ M⁻¹, respectively. The K_{B} agrees well with those reported from bulk measurement. The inventors also determined the number of T-ZZ molecules on living cells prepared as described in FIGS. 5A and B, showing 4640 and 2687 molecules of T-ZZ on the surface of 40 living cells, respectively. Thus, on average, there are 119 and 67 molecules/cell, which creates coverage of 0.37 and 0.21 molecules/μm², respectively. The calculation details are described in Tables 1 and 2 of SI.

At higher AgMMUA-IgG nanoparticle concentrations (2.78, 5.56, and 11.12 nM), the inventors observed a decrease in the number of bound nanoparticles after the binding reaction reached equilibrium at 80-100 min. This interesting phenomenon was not observed at the lowest concentration (1.39 nM) of AgMMUA-IgG (FIG. 6A, a), nor at the lower coverage of T-ZZ (FIG. 6B, a), suggesting that, as more AgMMUA-IgG nanoparticles bound with T-ZZ on the living cell surface, it may become harder to distinguish two nearby nanoparticles within the spatial resolution of the CCD camera (single pixel, 65 nm) using optical microscopy or that the higher concentrations of bound T-ZZ may lead to dissociation. The results also suggest that a high coverage of bound nanoparticles on living cells may promote their tendency to aggregate. Effort has been made to distinguish multiple nanoparticles from a single nanoparticle within the spatial resolution of CCD camera (1-2 pixels) using the scattering intensity of single nanoparticles. However, the inventors found no evidence that multiple nanoparticles were present within the spatial limit of the CCD camera. Thus, the observation may be attributable to the dissociation of AgMMUA-IgG from T-ZZ. Studies are underway to further characterize the mechanism.

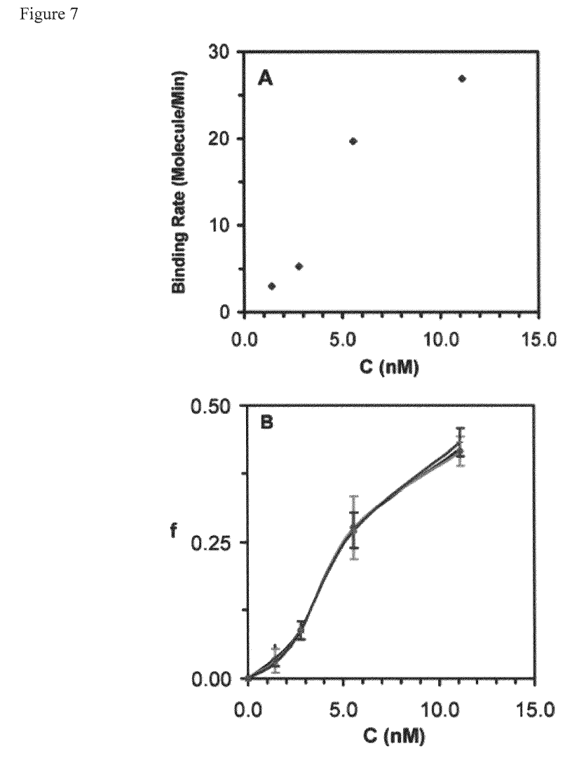

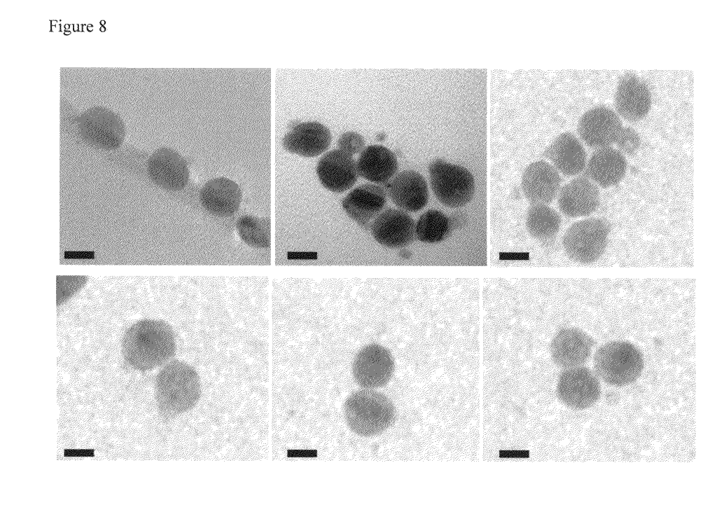

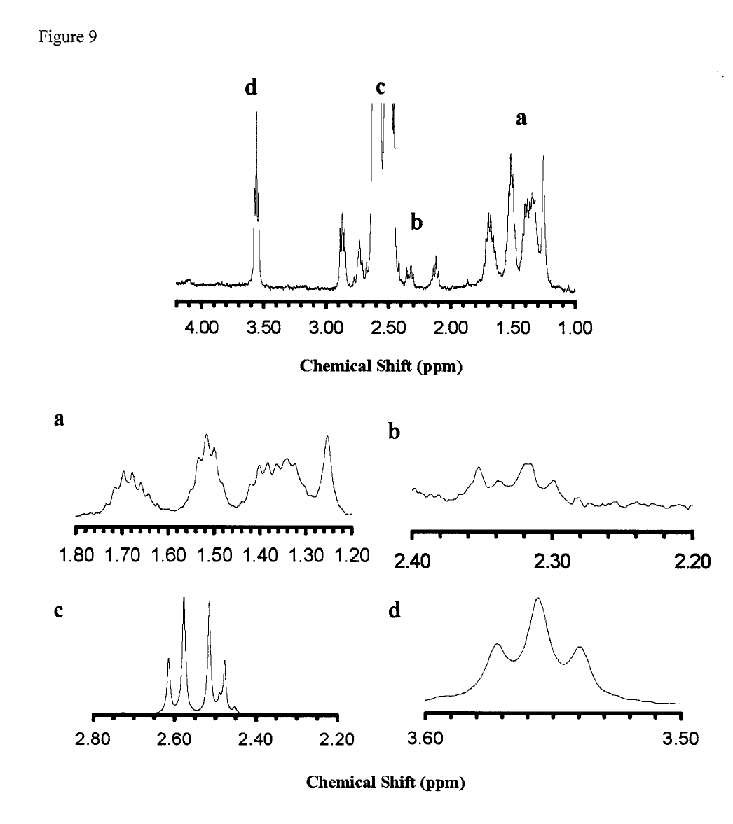

The binding rates of AgMMUA-IgG nanoparticles (1.39, 2.78, 5.56, and 11.12 nM) with T-ZZ on living cells were calculated from the slopes of FIG. 6A (a-d) within the first 60 min before reaching equilibrium, showing 3.0, 5.3, 20, and 27 molecules/min, respectively. A plot of the binding rates versus concentration of AgMMUA-IgG shows the high dependence of binding rates on concentration (FIG. 7A). Using the same approach, the inventors found that the binding rate for 2.78 nM AgMMUA-IgG nanoparticles with two different coverages of T-ZZ (0.21 and 0.37 molecules/μm²) in FIG. 6B is 2.3 and 5.3 molecules/min, respectively, showing the dependence of binding rate with the coverage of T-ZZ on living cells.

In accordance with the present invention, the inventors further analyzed the bound AgMMUA-IgG nanoparticles versus the concentration at binding equilibrium (80-120 min in FIG. 6A). Plots of fraction (f) of bound T-ZZ on living cells versus concentration of AgMMUA-IgG (isotherms in FIG. 7B) further indicate that the inventors detected T-ZZ molecules on living cells below their binding dissociation constant. Due to the limit of spatial resolution, higher concentrations of AgMMUA-IgG were not used to detect higher coverage of bound T-ZZ on living cells. Nevertheless, the inventors are able to calculate K_{B} as (4.8±2.0)×10⁷ M⁻¹ from the isotherms in FIG. 7B, which is similar to those calculated from FIG. 6A. The isotherms show a sigmoidal shape, suggesting that the binding reaction is cooperative, which means that T-ZZ molecules on the surface of living cells are not completely independent. In other words, the binding reaction of AgMMUA-IgG nanoparticles with individual T-ZZ molecules becomes more favorable in the presence of surrounding bound T-ZZ molecules. A plausible explanation would be that the presence of T-ZZ molecules bound with AgMMUA-IgG nanoparticles on the cell surface made it easier for other nanoparticles to dock onto the cell surface. Similar phenomena where bound nanoparticles on the cell surface serve as nucleation sites to help more nanoparticles to dock on the surface were also observed in our previous study.¹⁵

Taken together, the present invention shows that single-nanoparticle biosensors can be used to map low-coverage single receptor molecules (less than 50 receptors/cell) on single living cells in real time. Using optical microscopy, single-nanoparticle biosensors are not suitable for detecting high coverage of protein molecules on single living cells, because of limitation of optical resolution. Using TEM, one can expect to map high coverage of protein molecules on the cell surface using single-nanoparticle biosensors, but TEM cannot offer real-time kinetic measurements on living cells.

### Working Examples Part II

### A. Experimental Section

### Reagents and Supplies

Silver nitrate (≧299.9%), sodium borohydride (≧298%), sodium citrate dihydrate (≧299%), hydrogen peroxide (30%), polyvinylpyrrolidone (PVP), 11-mercaptoundecanoic acid (MUA≧95%), 6-mercapto-1-hexanol (MCH) (≧297%) and 30 kDa poly(ethylene glycol) (PEG) were purchased from Sigma-Aldrich. 1-Ethyl-3-[3-dimethylaminopropyl]-carbodiimide hydrochloride (EDC≧299%) and N-hydroxysulfosuccinimide (Sulfo-NHS≧98.5%) were purchased from Pierce. Recombinant human TNF-α (TNFSF1A) and monoclonal anti-human TNF-α antibody (TNFSF1A antibody) were purchased from R&D systems. All solutions, including 10 mM 6 Phosphate buffer saline (PBS) (pH=7.4, 10 mM of phosphate buffer and NaCl), were prepared using nanopure 18 MΩ deionized water (Bamstead).

### Synthesis and Characterization of MNOBS

### 2.6 nm Ag Nanoparticles

AgNO3 (0.11 mM), sodium citrate (1.91 mM), PVP (0.052 mM), and H2O2 (25.0 mM) in nanopure water (42.3 mL) were prepared freshly, mixed and stirred instantly. As NaBH4 (150 μL, 100 mM) was added into the mixture, the solution color turned to light yellow. After stirring for another 3 hr, the solution was filtered using 0.2 μm membrane filters. The Ag nanoparticle solution was immediately characterized using UV-vis spectroscopy Hitachi U3310), our darkfield single nanoparticle optical microscopy and spectroscopy SNOMS), and dynamic light scattering (DLS) (Nicomp 380ZLS particle sizing system). TEM samples were immediately prepared and further characterized using high-resolution transmission electron microscopy (HRTEM) (FEI Tecnai G2 F30 FEG).

In accordance with the present invention, the inventors characterized the photostability of single Ag nanoparticles by acquiring sequential optical images of single Ag nanoparticles using EMCCD camera with exposure time at 100 ms and readout time of 40.6 ms while these nanoparticles were constantly irradiated under dark-field microscope illuminator (100 W halogen) for 12 hr.24, 26, 27 The illumination power at the sample stage (focal plane of dark field) was (0.070±0.001) watt during the experiment. The inventors measured scattering intensity of individual nanoparticles and background over time.

### Functional Ag Nanoparticles

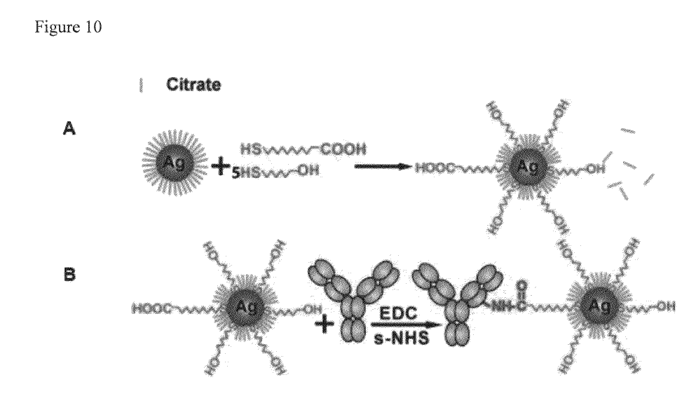

(AgMMUA) MUA (10 mM) and MCH (90 mM) in 0.5 mL ethanol were added to 50 mL of freshly prepared Ag nanoparticle aqueous solution and stirred for 24 hr to attach MUA and MCH onto the surface of 7 nanoparticles via their interaction of —SH groups with nanoparticles (FIG. 10). The AgMMUA nanoparticles were washed twice to remove excess thiol using centrifugation (Beckman Optima L90k, 50 Ti rotor, 30,000 rpm at 4° C. for 60 min). The AgMMUA nanoparticle solutions were immediately characterized using UV-vis spectroscopy, SNOMS, and DLS. NMR samples were immediately prepared by washing AgMMUA nanoparticles with nanopure water three times using centrifugation, drying the samples using lyophilize (VirTis) and dissolving 50 mg of AgMMUA nanoparticles in 1 mL D2O, and further characterized by NMR (400 MHz, Bruker).

### AgMMUA-MAB Nanoparticles (SMNOBS)

In accordance with the present invention, a two-step method was used to conjugate the carboxyl group of AgMMUA with amine group of MAB via a peptide bond using EDC and sulfo-NHS as mediators (Scheme 1B). EDC (77 mol) and sulfo-NHS (7.7 mol) were added to AgMMUA aqueous solution (50 mL, 154 nM), forming AgMMUA-s-NHS esters. After stirring at room temperature for 40 min, AgMMUA-s-NHS was desalted using a Centriprep YM-30 (Millipore) by centrifugation at 1500 ruff (relative-centrifuge force) for 5 min to remove excess EDC and sulfo-NHS, and then re-dissolved in 10 mM phosphate buffer (PB). PEG (0.05% w/v) was added to prevent nonspecific adsorption of MAB onto the surface of nanoparticles. In the second step, MAB was added to 50 mL AgMMUA-s-NHS in 10 mM PB solution at a mole ratio of MAB to AgMMUA of 0.97. The solution was mixed using a rotary shaker at room temperature for 2 hr and then at 4° C. for 12 hr. The final product (AgMMUA-MAB nanoparticles) was washed using 10 mM PBS buffer to remove excess MAB using centrifugation with 30,000 rpm at 4° C. for 60 min. The pellet was resuspended in 10 mM PBS with 0.05% w/v PEG, and stored at 4° C. for the future use. The size and optical properties of AgMMUA-MAB nanoparticles were characterized using UV-vis spectroscopy, SNOMS and DLS.

### Bioactivity, Sensitivity, Specificity, and Dynamic Range of SMNOBS

### Bulk Analysis of TNF-α in Solution

In accordance with the present invention, both TNF-α and AgMMUA-MAB solutions were prepared in 10 mM PBS buffer. The UV-vis spectra of the mixture (AgMMUA-MAB nanoparticles incubated with TNF-α) were measured over 48 hr to monitor the binding kinetics and affinity of AgMMUA-MAB with TNF-α. The spectra were measured at room temperature every 5 min for the first 2 hr and at every 2.5 hr after that until 48 hr. The mixture was stored at 4° C. to prevent denaturation of the proteins between spectroscopy measurements. The AgMMUA-MAB nanoparticles (50 nM) were used to detect several concentrations of TNF-α solution (1, 10, 100, 200 ng/mL) to construct a calibration curve and to determine the dynamic range of SMNOBS. Blank control experiments were carried out by replacing BSA with TNF-α, incubating BSA with AgMMUA-MAB, and measuring UV-vis spectra of the mixture over time.

### Real-Time SMD of TNF-α

In accordance with the present invention, single AgMMUA-MAB nanoparticles were placed on a clean slide by incubating 2 μL of 50 nM AgMMUA-MAB on the slide for 5-10 min, removing the solution by a pipette, and thoroughly rinsing the surface of slide using PBS buffer. The self-made microchamber was created on the slide, allowing AgMMUA-MAB nanoparticles to be incubated with 10 μL PBS buffer. Dark-field optical images and spectra of single AgMMUA-MAB nanoparticles were acquired using SNOMS. TNF-α solution (10 μL, 10 ng/mL) was carefully injected into the microchamber and timer was simultaneously started. Dark-field optical images and spectra of single AgMMUA-MAB nanoparticles were acquired using SNOMS in real time at every 10 min for 8 hr as the single MAB molecules attached onto the nanoparticles bound with single TNF-α molecules in solution. The inventors carried out blank control experiments to characterize specificity and selectivity of SMNOBS by replacing TNF-α with BSA, incubating BSA with single AgMMUA-MAB nanoparticles, and acquiring dark-field optical images and spectra of single AgMMUA-MAB nanoparticles for 8 hr using SNOMS.

In accordance with the present invention, SNOMS used in previous studies for real-time imaging and spectroscopic characterization of single nanoparticles in/on single living cells and in zebrafish embryos, and for SMD.24-“ ” In this study, Nuance Multispectral Imaging System (Cambridge Research & Instrumentation) and other detectors (EMCCD, 5 MHz Micromax CCD camera and color digital camera) coupled with a SpectraPro-150 (Roper Scientific) were used for imaging and measuring localized surface plasmon resonance spectra (LSPRS) of single nanoparticles (Ag, AgMMUA, AgMMUA-MAB, AgMMUA-MAB-TNF-α).

### Statistical Analysis

In accordance with the present invention, the inventors investigated over 100 nanoparticles for each measurement. At least three measurements were performed in each solution. Thus, at least 300 nanoparticles in each solution were studied to gain sufficient statistics to determine their size distribution and color distribution that represented the bulk solution at the single nanoparticle level. All other measurements, including characterization of photostability and LSPR spectra of single nanoparticles and control experiments, were repeated at least three times.

### Synthesis and Characterization of SMNOBS

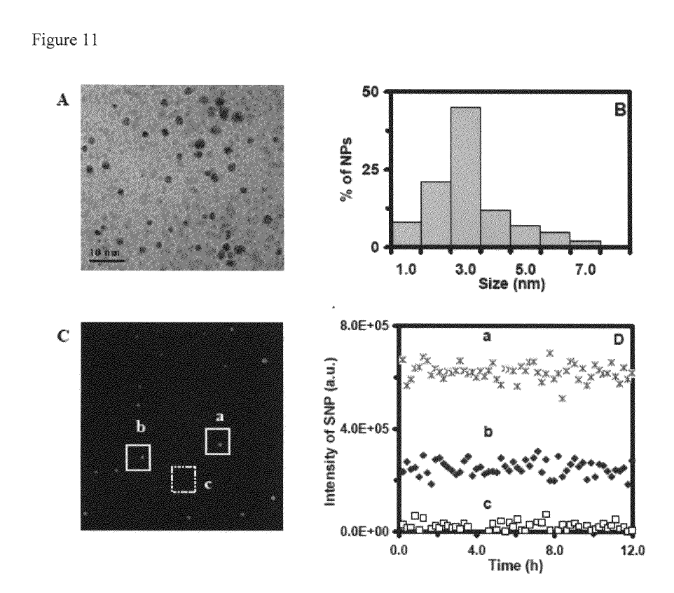

In accordance with the present invention, the inventors synthesized small, stable Ag nanoparticles by reducing AgNO3 with sodium citrate and hydrogen peroxide in the presence of a stabilizer (PVP), and characterized these nanoparticles using HRTEM, showing that they were nearly sphere with a diameter of 2.6±1.0 nm (86% of 1-4 nm and 14% of 5-7 nm) (FIGS. 11A & B). The nanoparticles were stable in solution (nonaggregation) for months, suggesting that PVP and citrate played a role in preventing nanoparticles from aggregation.

In accordance with the present invention, the inventors characterized the photostability of single Ag nanoparticles by acquiring sequential optical images of single Ag nanoparticles while these nanoparticles were constantly irradiated under dark-field microscope illuminator (100 W halogen) over 12 hr. The inventors measured scattering intensity of individual nanoparticles within a 20×20 pixel area (squared in FIG. 11C: a & b) and average background intensity of several detection areas with the same size of detection volume (20×20 pixel) in the absence of nanoparticles (squared in FIG. 11C: c). The inventors subtracted the average background intensity from the integrated intensity of single nanoparticles and individual background area and plotted the subtracted integrated intensity of individual nanoparticles and background as a function of time (FIG. 11D). The plots show that the scattering intensity of single nanoparticles remained constant over time and slight fluctuations of scattering intensity of single nanoparticles were similar to those observed from background, suggesting that these fluctuations might be attributable to intensity fluctuation of microscope illuminator or dark noise of EMCCD camera. Thus, the results demonstrate that single nanoparticles are photostable and do not suffer photodecomposition and blinking.

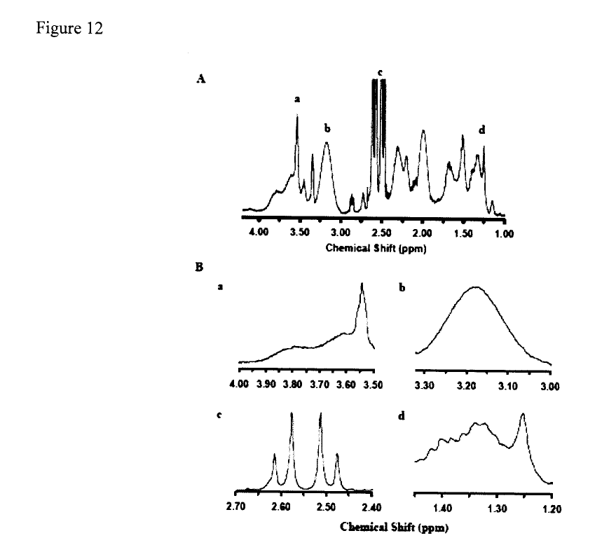

To prepare AgMMUA in accordance with the present invention, the inventors incubated the Ag nanoparticles with high concentrations of MCH and MUA and attached a monolayer of mixed MUA and MCH onto Ag nanoparticles by the interaction of their thiol groups (—SH) with the surface of nanoparticles and via replacement reactions of citrate groups with MUA and MCH (FIG. 10). The inventors used NMR to characterize the functional groups on the surface of AgMMUA nanoparticles and found that the molar ratio of attached functional groups of citrate: MCH:MUA:PVP was 28:5:1:0.04 (FIG. 12), showing that MCH and MUA successfully replaced some of the citrate groups on the surface of nanoparticles and part of PVP molecule was also attached on the nanoparticles. With such a molar ratio of attached functional groups, the inventors calculated the number of functional molecules that could be attached on the surface of a single nanoparticle (3 nm in diameter; surface area=28.3 nm2), using a close-packing model and found that approximately only a single MUA (0.9) molecule was physically possible to be attached on a single Ag nanoparticle. Note that the cross-sectional areas of a citrate, MUA and MCH molecule are 1.13, 4.84×10⁻² and 4.84×10⁻² nm², respectively.

In accordance with the present invention, the inventors then conjugated the amine group of MAB with the carboxyl group of MUA attached on Ag nanoparticles via a peptide bond using EDC and sulfo-NHS as mediators to prepare AgMMUAMAB nanoparticles (SMNOBS) (FIG. 10B). Since each nanoparticle was attached with a single MUA molecule, it ensured that a single MAB molecule was tagged with a single nanoparticle. Even if more than one MUA molecules were attached on the surface of a single nanoparticle, the small size of nanoparticles would allow only a single MAB molecule to be conjugated with the attached MUA molecules because of steric effects (insufficient space to accommodate more than a single bulky MAB molecule on the surface of a 2.6 nm Ag nanoparticle). Thus, using the small surface area of a single nanoparticle, the inventors effectively created a SMD volume that allowed one molecule of MAB to be conjugated with a single nanoparticle and successfully prepared a SMNOBS for sensing a single TNF-α molecule.

### Effects of Functional Groups on Size and Optical Properties of Single Nanoparticles

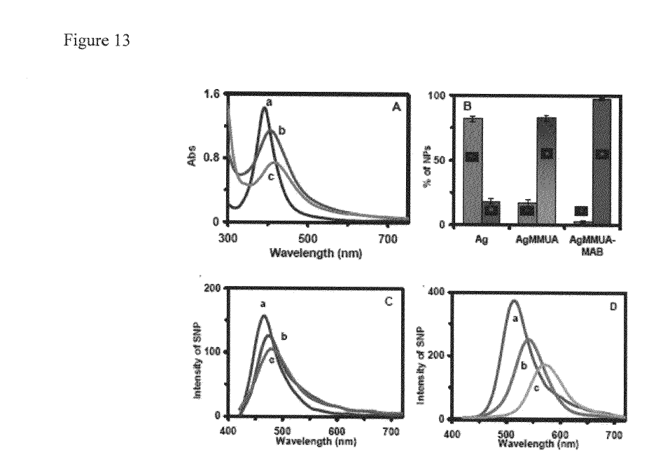

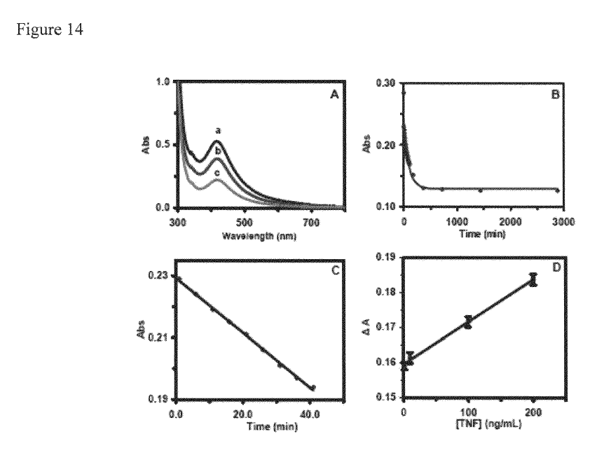

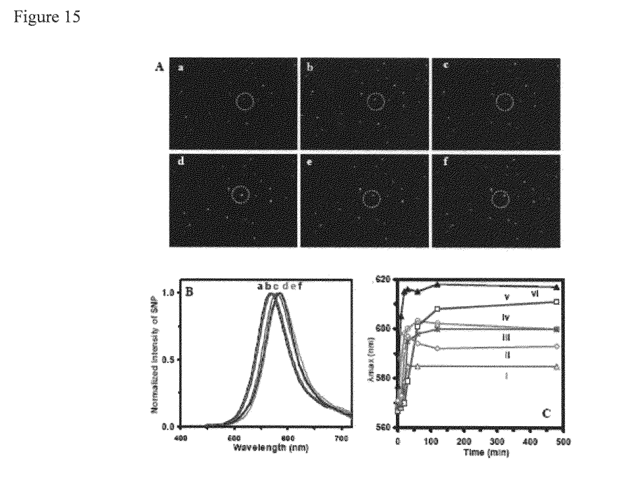

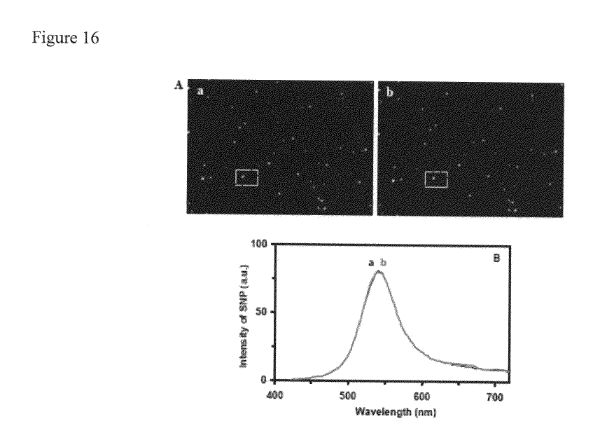

UV-vis spectra of Ag, AgMMUA and AgMMUA-MAB nanoparticle solutions in FIG. 13A show that the spectra, in accordance with the present invention, became broader with the absorbance decreased and the peak wavelength shifted to the longer wavelength (red-shift), as Ag nanoparticles were functionalized with monolayer of mixed MUA and MCH, and as AgMMUA was conjugated with MAB. The changes of absorption spectra (e.g., peak wavelength, absorbance and FWHM) of 2.6 nm Ag nanoparticles as the nanoparticles were functionalized were much greater (at least twice) than those the inventors reported previously in 12 nm Ag nanoparticles.^{B24} This result suggests that the smaller Ag nanoparticles exhibited higher dependence of optical properties on surface properties (surface functional groups), which may be attributable to the higher surface-area-to-volume ratio of smaller nanoparticles. Thus, the smaller nanoparticles are more sensitive to the change of surface functional groups, making it a much more sensitive biosensor.

In accordance with the present invention, using DLS, the inventors found that the size distributions of Ag, AgMMUA and AgMMUA-MAB nanoparticles suspended in the solution were 2.6±1.1, 3.6±1.5, and 18.6±5.2, respectively. The results are in excellent agreement with those computed using modeling systems (Table I), further demonstrating that AgMMUA and AgMMUA-MAB were successfully prepared.

TABLE I
Characterization of Size and Optical Properties of Ag
AgMMUA and AgMMUA-MAB Nanoparticles.

| | | | LSPRS of Single | | | |
|---|---|---|---|---|---|---|
| | *Diameter of | | NP (nm) | | | |
| | Bulk NPs (nm) | | Blue | | Green | |
| Nano- | Experi- | Theo- | λmax | FWHM | λmax | FWHM |
| particles | mental | retical | (nm) | (nm) | (nm) | (nm) |
| Ag | 2.6 ± 1.1 | N/A | 465 ± 3 | 52 ± 3 | 513 ± 6 | 63 ± 5 |
| AgMMUA | 3.6 ± 1.5 | 4.5 | 474 ± 6 | 64 ± 4 | 543 ± 7 | 69 ± 6 |
| AgMMUA- | 18.6 ± 5.2 | 16.6 | 477 ± 7 | 77 ± 6 | 570 ± 9 | 73 ± 8 |
| MAB | | | | | | |

*Experimental measurement and theoretical calculation of average diameters of bulk nanoparticles. Note that lengths of MUA, MCH and MAB (anti-TNF-α) are 1.521, 0.959 and 12.1 nm, calculated using ChemSketch 3D view and protein data bank, respectively.

Histograms of distribution of colors (LSPRS) of single nanoparticles in the solution (FIG. 13B: Ag) illustrate a nearly uniform Ag nanoparticle solution with (82±2) % of nanoparticles being blue (λmax=465±3 nm) and (18±2) % of them being green (λmax=513±6 nm) (FIGS. 13C & D: a), respectively. As MUA and MCH functional groups replaced citrate groups (FIG. 13B: AgMMUA), colors (LSPRS) of single nanoparticles shifted to the longer wavelength and (83±2) % of nanoparticles became light green (λmax=543±7 nm; FIG. 13D: b) with (17±2) % of them remaining blue (λmax=474±6 nm; FIG. 3C: b), suggesting that majority of Ag nanoparticles were functionalized with MUA and MCH. As AgMMUA nanoparticles were conjugated with MAB (FIG. 13B: AgMMUA-MAB), color of individual nanoparticles shifted further to the longer wavelength and (97±1) % of nanoparticles became dark green (λmax=570±9 nm; FIG. 13D: c) with only (3±1) % of them remaining blue (λmax=457±7 nm; FIG. 13C: c), indicating a nearly complete conjugation reaction.

Representative LSPR spectra of single Ag, AgMMUA and AgMMUA-MAB nanoparticles are shown in FIGS. 13C & D and the quantitative changes of peak wavelength and FWHM are summarized in Table I. In accordance with the present invention, the scattering intensity of single blue Ag nanoparticles (FIG. 13C: a) is lower than that of the green nanoparticle (FIG. 13D: a), suggesting that the blue nanoparticle is smaller than the green nanoparticle, which shows size-dependent colors (LSPRS) of single nanoparticles and agrees with what the inventors observed previously. Notably, the FWHM of single blue Ag nanoparticle (52±3 nm) were 11 nm smaller than that of single green Ag nanoparticles (63±5 nm), suggesting that the smaller size (1-4 nm in diameter) of single blue nanoparticles resulted in a shorter dephasing time, T2, than that of slightly larger nanoparticles (5-7 nm) of green nanoparticles.^{B33}

### Bioactivity, Specificity and Dynamic Range of SMNOBS

In accordance with the present invention, the inventors characterized the bioactivity of AgMMUA-MAB nanoparticles by measuring their binding affinity with TNF-α and comparing it with those reported in the literature. UV-vis spectra of AgMMUA-MAB nanoparticles exhibited stable peak wavelengths at 417±3 nm (FIG. 14A: a) with the extinction coefficient (molar absorptivity, ∈) of 4.6×10⁶ M⁻¹cm⁻¹, which was determined by the Beer-Lambert Law. As AgMMUA-MAB nanoparticles were incubated with TNF-α, absorbance of the spectra decreased over incubation time with its peak wavelength (417 nm) and FWHM remaining essentially unchanged (FIG. 14A), suggesting that AgMMUA-MAB nanoparticles bound with TNF-α and the bound AgMMUA-MAB-TNF-α nanoparticles did not contribute to the absorption, which might be attributable to precipitation of nanoparticles from the solution due to the cross-linking of nanoparticles.^{B24} A plot of the peak absorbance subtracted from baseline versus time in FIG. 4B exhibited a high linearity during the first 40 min of the incubation time and then remained constant, suggesting that the binding of AgMMUA-MAB with TNF-α was a first-order reaction as illustrated in Eq. 1:

AgMMUA-MAB+TNF-α□AgMMUA-MAB-TNF-α [3]

Note that TNF-α concentration (500 nM) was 10 times higher than that of AgMMUA-MAB (50 nM). Thus, TNF-α concentration remained essentially unchanged over the entire reaction, and a second-order (or multiple-order) reaction can be treated as a pseudo-first-order reaction. From the decrease of peak absorbance (ΔA=∈bC; where b is the light path and C is nanoparticle concentration) in FIG. 14B, the inventors found that 25.6 nM AgMMUA-MAB bound with TNF-α as the binding reaction reached the equilibrium. Therefore, the inventors determined the equilibrium concentrations of AgMMUA-MAB (24.4 nM), TNF-α (474.4 nM) and AgMMUA-ABTNF-α (25.6 nM) and used them to calculate the equilibrium constant (affinity constant, KB) of AgMMUA-MAB with TNF-α as (2.2±0.1)×10⁶ M⁻¹.

In accordance with the present invention, the inventors also calculated the association (binding) (kₐ) and dissociation (k_{d}) rate constants as 7.7×10³ and 7.1×10³ M⁻¹min⁻¹ (FIG. 14C), respectively, by fitting the experimental data (points) with simulation of binding reaction (line) using the equation, A=0.13+0.11e−0.011t, which was derived using the same approach as the inventors described previously.^{B24} Here A and t represent absorbance and reaction time, respectively. From the association (kₐ) and dissociation (k_{d}) rate constants, the inventors also determined the binding affinity constant, K_{B}=kₐ/k_{d}=(1.1±0.1)×10⁶ M⁻¹, which is close to that measured directly using equilibrium concentrations.

In accordance with the present invention, the average of K_{B} is (1.7±0.1)×10⁶ M⁻¹, which agrees well with the affinity constant (1.8×10⁶ M⁻¹) of TNF-α with a monoclonal antibody reported previously using other methods.^{B34} Even though the binding sites and MAB in the reported study might differ from ours, it offers an effective reference for comparison. Thus, the result suggests that MAB attached on AgMMUAMAB nanoparticles remained its biological activities and steric effect of functional nanoparticles did not significantly interfere the binding of attached MAB with TNF-α in solution.

In accordance with the present invention, the inventors then investigated the calibration curves of SMNOBS (AgMMUA-MAB) by plotting the decrease peak absorbance of AgMMUA-MAB (ΔA) in response to the presence of various concentrations of TNFα (versus concentrations of TNFα: 0, 1, 10, 100, and 200 ng/mL in 10 mM PBS solution), illustrating a linear calibration curve in FIG. 14D. The result shows that ΔA is proportional to the concentration of TNFα. The inventors performed blank control experiments by replacing TNFα with the same concentrations of BSA in PBS buffer and using the same volume of PBS buffer only (in the absence of TNFα). The inventors found that AA were independent of BSA concentration, and remained essentially unchanged with a slight decrease in absorbance, owing to the dilution of AgMMUA-MAB by addition of PBS buffer. The result shows high specificity and selectivity of SMNOBS, and exhibits a dynamic range of at least 0-200 ng/mL. Since the inventors are much more interested in sensing low concentrations of TNFα using SMNOMS, the inventors did not use higher concentrations of TNFα beyond 200 ng/mL. Previous studies reported that TNFα concentrations in normal arterial plasma of health human beings ranged from 1.2 to 130 pg/mL and many of these measurements were highly dependent upon the sensitivity of assays.^{B6, 7,35} A variety of diseases, such as cancer, diabetes, coronary artery disease, and HIV infection, could increase TNFα concentration 100-1000 folds higher than its normal level,^{B4, 35, 36} which is within the dynamic range of our nanoparticle biosensors. Thus, SMNOBS of the present invention are well suited for monitoring disease diagnosis and therapy.

### Real-time Sensing of Single TNF-α Molecules and its Binding Reactions

In accordance with the present invention, the inventors further characterized the detection sensitivity of SMNOBS and used them to detect single TNFα molecules in solution in real time. Dark-field images of single AgMMUA-MAB nanoparticles incubated with TNFα molecules in PBS buffer solution over 8 hr showed the colors of single AgMMUA-MAB nanoparticles changed from green to yellowish green over time (FIG. 15A: a-f) and the peak wavelength of their LSPR spectra shifted to the longer wavelength with the average of peak wavelength shifts (Δλmax) at 29±11 nm (FIGS. 15B & C). The LSPR spectra of the representative nanoparticle circled in FIG. 15A showed a red shift of peak wavelength from 543 (green) (FIG. 15B: a) to 572 nm (FIG. 15B: d-f), as the nanoparticle was incubated with TNFα molecules, illustrating the binding reaction reached its equilibrium at 30-60 min (FIG. 15B: d-e & 5C: a). Plots of peak wavelength (λmax) of LSPRS of single nanoparticles versus incubation time in FIG. 15C showed that SM binding reactions of TNFα with MAB attached on single nanoparticles reached equilibrium at various times, ranging from 30 to 120 min, which suggested stochastic binding kinetics of SM specific interactions.

In accordance with the present invention, the peak wavelength shift (29±11 nm) of single AgMMUA-MAB nanoparticles exhibits unprecedentedly high sensitivity of single nanoparticle biosensors for detecting single protein molecules (e.g., TNFα). The red shift of peak wavelength might be attributable to the lower LSPR energy, owing to (i) chemical interface damping and the change of dielectric constants and surrounding environments of the nanoparticles^{22, 37} and (ii) an increase in the effective radius and aspect ratio of single nanoparticles²² as NFα molecules diffused to the surface of nanoparticles and the binding of a single TNFα molecule with a single MAB attached on a nanoparticle occurred. The inventors carried out blank control experiments by replacing TNFα with BSA in the experiment and the inventors did not observe any significant shift of LSPRS of single AgMMUA-MAB nanoparticles (FIG. 16), showing the high selectivity and specificity of SMNOBS. Note that the red shift of peak wavelength was not caused by the aggregation of nanoparticles because the nanoparticles were coated on the slide and thoroughly rinsed using PBS buffer before incubating with TNFα solution and there were no nanoparticles in solution that might aggregate with AgMMUA-MUA nanoparticles on the surface. Thus, the results showed the possibility of using LSPRS of single nanoparticles to monitor SM reactions on the surface of single nanoparticles.

Taken together, the inventors have demonstrated that AgMMUA-MAB nanoparticles can be used as SMNOBS to detect and image single TNFα molecules and their binding reactions at the single molecule level in real time with high sensitivity and selectivity. The inventors found that the binding kinetics of SM binding reactions varied from one single molecule to the other, and from a SMNOBS to the other, illustrating the range of equilibrium time from 30 to 120 min.

### REFERENCES

### “A Series”

(A1) Gurevich, K. G.; Agutter, P. S.; Wheatley, D. N. Cell Signal 2003, 15, 447-453.
(A2) Williamson, S. A.; Knight, R. A.; Lightman, S. L.; Hobbs, J. R. Brain, Behav., Immun. 1987, 1, 329-335.
(A3) Williamson, S. A.; Knight, R. A.; Lightman, S. L.; Hobbs, J. R. Immunology 1988, 65, 47-51.
(A4) Dower, S. K.; Kronheim, S. R.; Hopp, T. P.; Cantrell, M.; Deeley, M.; Gillis, S.; Henney, C. S.; Urdal, D. L. Nature 1986, 324, 266-268.
(A5) Weiss, S. Science 1999, 283, 1676-1683.
(A6) Janshoff, A.; Neitzert, M.; Oberdorfer, Y.; Fuchs, H. Angew. Chem., Int. Ed. Engl. 2000, 39, 3212-3237.
(A7) Beesley, J. E. Colloidal Gold: A New Perspective for Cytochemical Marking; Oxford University Press: Royal Microscopy Society, 1989; p 25.
(A8) Manweiler, K.; Hohenberg, H.; Bohn, W.; Rutter, G. J. Microsc. 1982, 126, 145-149.
(A9) Haes, A. J.; Van Duyne, R. P. J. Am. Chem. Soc. 2002, 124, 10596-10604.
(A10) Lyon, L. A.; Musick, M. D.; Natan, M. J. Anal. Chem. 1998, 70, 5177-5183.
(A11) Nam, J. M.; Thaxton, C. S.; Mirkin, C. A. Science 2003, 301, 1884-1886.
(A12) Schultz, S.; Smith, D. R.; Mock, J. J.; Schultz, D. A. Proc. Natl. Acad. Sci. U.S.A. 2000, 97, 996-1001.
(A13) Kyriacou, S. V.; Brownlow, W. J.; Xu, X. H. N. Biochemistry 2004, 43, 140-147.
(A14) Sandhu, K. K.; McIntosh, C. M.; Simard, J. M.; Smith, S. W.; Rotello, V. M. Bioconjugate Chem. 2002, 13, 3-6.
(A15) Xu, X. H. N.; Chen, J.; Jeffers, R. B.; Kyriacou, S. V. Nano Lett. 2002, 2, 175-182.
(A16) Xu, X. H. N.; Brownlow, W. J.; Kyriacou, S. V.; Wan, Q.; Viola, J. J. Biochemistry 2004, 43, 10400-10413.
(A17) Xu, X. H. N.; Song, Y.; Nallathamby, P. D. Probing membrane transport of single live cells using single molecule detection and single nanoparticle assay. In Ultrasensitive Bioanalysis: Advanced Analytical Chemistry Applications in Nanobiotechnology, Single Molecule Detection, and Single Cell Analysis; Xu, X. H. N., Ed.; Wiley: Hoboken, N.J., 2007; pp 41-70.
(A18) Haes, A. J.; Zhao, J.; Zou, S.; Own, C. S.; Marks, L. D.; Schatz, G. C.; Van Duyne, R. P. J. Phys. Chem. B 2005, 109, 11158-11162.
(A19) Ambrose, W. P.; Goodwin, P. M.; Jett, J. H.; Van Orden, A.; Werner, J. H.; Keller, R. A. Chem. Rev. 1999, 99, 2929-2956.
(A20) Nie, S.; Zare, R. N. Annu. Rev. Biophys. Biomol. Struct. 1997, 26, 567-596. (A21) Xu, X. H.; Yeung, E. S. Science 1997, 275, 1106-1109.
(A22) Xu, X. H. N.; Yeung, E. S. Science 1998, 281, 1650-1653.
(A23) Xu, X. H. N.; Jeffers, R. B.; Gao, J.; Logan, B. Analyst 2001, 126, 1285-1292.
(A24) Byassee, T. A.; Chan, W. C.; Nie, S. Anal. Chem. 2000, 72, 5606-5611.
(A25) Xu, X. H. N.; Brownlow, W. J.; Huang, S.; Chen, J. Biochem. Biophys. Res. Commun. 2003, 305, 79-86.
(A26) Yu, J.; Xiao, J.; Ren, X.; Lao, K.; Xie, X. Science 2006, 311, 1600-1603.
(A27) Kyriacou, S. V.; Nowak, M. E.; Brownlow, W. J.; Xu, X. H. N. J. Biomed. Opt. 2002, 7, 576-586.
(A28) Chenal, A.; Savarin, P.; Nizard, P.; Guillain, F.; Gillet, D.; Forge, V. J. Biol. Chem. 2002, 277, 43425-43432.
(A29) Nizard, P.; Chenal, A.; Beaumelle, B.; Fourcade, A.; Gillet, D. Protein Eng. 2001, 14, 439-446.
(A30) Nizard, P.; Liger, D.; Gaillard, C.; Gillet, D. FEBS Lett. 1998, 433, 83-88.
(A31) Oh, K. J.; Zhan, H.; Cui, C.; Hideg, K.; Collier, R. J.; Hubbell, W. L. Science 1996, 273, 810-812.
(A32) Montagner, C.; Perier, A.; Pichard, S.; Vernier, G.; Menez, A.; Gillet, D.; Forge, V.; Chenal, A. Biochemistry 2007, 46, 1878-1887. (33) Nizard, P.; Gross, D. A.; Babon, A.; Chenal, A.; Beaumelle, B.; Kosmatopoulos, K.; Gillet, D. J. Immunother. 2003, 26, 63-71.
(A33) Nizard, P.; Gross, D. A.; Babon, A.; Chenal, A.; Beaumelle, B.; Kosmatopoulos, K.; Gillet, D. J. Immunother. 2003, 26, 63-71.
(A34) Choe, S.; Bennett, M. J.; Fujii, G.; Curmi, P. M.; Kantardjieff, K. A.; Collier, R. J.; Eisenberg, D. Nature 1992, 357, 216-222.
(A35) Xu, X. H. N.; Huang, S.; Brownlow, W.; Salatia, K.; Jeffers, R. J. Phys. Chem. B. 2004, 108, 15543-15551.
(A36) Xu, X. H. N.; Wan, Q.; Kyriacou, S. V.; Brownlow, W. J.; Nowak, M. E. Biochem. Biophys. Res. Commun. 2003, 305, 941-949.
(A37) Li, X.; Xu, W.; Zhang, J.; Jia, H.; Yang, B.; Zhao, B.; Li, B.; Ozaki, Y. Langmuir 2003, 19, 4285-4290.
(A38) Panigrahi, S.; Praharaj, S.; Basu, S.; Ghosh, S. K.; Jana, S.; Pande, S.; Vo-Dinh, T.; Jiang, H.; Pal, T. J. Phys. Chem. B 2006, 110, 13436-13444.
(A39) Yamamoto, M.; Kashiwagi, Y.; Nakamoto, M. Langmuir 2006, 22, 8581-8586.
(A40) Laibinis, P. E.; Hickman, J. J.; Wrighton, M. S.; Whitesides, G. M. Science 1989, 245, 845-847.
(A41) Lee, J. K.; Yoon, T. J.; Chung, Y. K. Chem. Commun. 2001, 1164-1165.
(A42) Wawer, I.; Pisklak, M.; Chilmonczyk, Z. J. Pharm. Biomed. Anal. 2005, 38, 865-870.
(A43) Hasan, M.; Bethell, D.; Brust, M. J. Am. Chem. Soc. 2002, 124, 1132-1133.
(A44) Ingram, R. S.; Hostetler, M. J.; Murray, R. W. J. Am. Chem. Soc. 1997, 119, 9175-9178.
(A45) Kohlmann, O.; Steinmetz, W. E.; Mao, X. A.; Wuelfing, W. P.; Templeton, A. C.; Murray, R. W.; Johnson, C. S. J. Phys. Chem. B 2001, 105, 8801-8809.
(A46) Mie, G.; Beitra”g, G. Ann. Phys. 1908, 25, 377-445.
(A47) Ghetie, V.; Fabricius, H. A.; Nilsson, K.; Sjoquist, J. Immunology 1974, 26 (6), 1081-1091.
(A48) Hjelm, H.; Sjodahl, J.; Sjoquist, J. Eur. J. Biochem. 1975, 57 (2), 395-403.
(A49) Langone, J. J. Protein A of Staphylococcus aureus and Related Immunoglobulin Receptors Produced by Streptococci and Pneumonococci. In Advances in Immunology; Academic Press: New York, 1982, p 207.
(A50) Lancet, D.; Isenman, D.; Sjo”dahl, J.; Sjo”quist, J.; Pecht, I. Biochem. Biophys. Res. Commun. 1978, 85, 608-614.
(A51) Tinoco, I.; Sauer, K.; Wang, J.; Puglisi, J. D. In Physical Chemistry-Principles and Applications in Biological Sciences; Prentice Hall: Upper Saddle River, N.J., 2002; pp 315-387.
(A52) Jansson, B.; Uhlen, M.; Nygren, P. A. FEMS Immunol. Med. Microbiol. 1998, 20, 69-78.

### REFERENCES

### “B Series”

(B1) Nicola, N. A. Guidebook to Cytokines and Their Receptors; A Sambrook & Tooze Publication at Oxford University Press: Oxford, 1994.
(B2) Gurevich, K. G.; Agutter, P. S.; Wheatley, D. N. Cell Signal 2003, 15, 447-453.
(B3) Tagawa, M. Curr. Pharm. Des. 2000, 6, 681-699.
(B4) Haworth, C.; Maini, R. N.; Feldman, M. In The Cytokine Handbook; Thomson, A., Ed.; Academic Press, 1998, pp 777-801, and references therein.
(B5) Copeland, S.; Siddiqui, J.; Remick, D. J. Immunol. Methods 2004, 284, 99-106.
(B6) Klabusay, M.; Kohutova, V.; Coupek, P.; Nenickova, M.; Tesarova, E. Mediators of Inflammation 2006, 1-7.
(B7) de Kossodo, S.; Houba, V.; Grau, G. E. J. Immunol. Methods 1995, 182, 107-114.
(B8) Watts, A. D.; Onier-Chemx, N.; Hunt, N. H.; Chaudhri, G. J. Immunol. Methods 1999, 225, 179-184.
(B9) Shinn, A. H.; Bravo, N. C.; Maecker, H. T.; Smith, J. W. J. Immunol. Methods 2003, 282, 169-174.
(B10) Pico de Coana, Y.; Barrero, C.; Cajiao, I.; Mosquera, C.; Patarroyo, M. E.; Patarroyo, M. A. Cytokine 2004, 27, 129-133, and references therein.
(B11) Zajac, A.; Song, D.; Qian, W.; Zhukov, T. Colloids Surf. B. Biointerfaces 2007, 58, 309-314.
(B12) Wang, J.; Liu, G.; Engelhard, M. H.; Lin, Y. Anal. Chem. 2006, 78, 6974-6979.
(B13) Haes, A. J.; Van Duyne, R. P. J. Am. Chem. Soc. 2002, 124, 10596-10604.
(B14) Nam, J. M.; Thaxton, C. S.; Mirkin, C. A. Science 2003, 301, 1884-1886.
(B15) Agrawal, A.; Sathe, T.; Nie, S. In New Frontiers in Ultrasensitive Bioanalysis: Advanced Analytical Chemistry Applications in Nanobiotechnology, Single Molecule Detection, and Single Cell Analysis; Xu, X.-H. N., Ed.; Wiley: New Jersey, 2007, pp 71-89, and references therein.
(B16) Schultz, S.; Smith, D. R.; Mock, J. J.; Schultz, D. A. Proc. Natl. Acad. Sci. U.S.A. 2000, 97, 996-1001.
(B17) Xu, X.-H. N.; Song, Y.; Nallathamby, P. D. In New Frontiers in Ultrasensitive Bioanalysis: Advanced Analytical Chemistry Applications in Nanobiotechnology, Single Molecule Detection, and Single Cell Analysis; Xu, X.-H. N., Ed.; Wiley: New Jersey, 2007, pp 41-65, and references therein.
(B18) Beveridge, A. C.; Jett, J. H.; Keller, R. A. In New Frontiers in Ultrasensitive Bioanalysis: Advanced Analytical Chemistry Applications in Nanobiotechnology, Single Molecule 20 Detection, and Single Cell Analysis; Xu, X.-H. N., Ed.; Wiley: New Jersey, 2007, pp 1-27, and references therein.
(B19) Darzacq, X.; Singer, R.; Shav-Tal, Y. In New Frontiers in Ultrasensitive Bioanalysis: Advanced Analytical Chemistry Applications in Nanobiotechnology, Single Molecule Detection, and Single Cell Analysis; Xu, X.-H. N., Ed.; Wiley: New Jersey, 2007, pp 29-39, and references therein.
(B20) Xu, X.-H. N.; Yeung, E. S. Science 1997, 275, 1106-1109.
(B21) Bohren, C. F.; Huffman, D. R. Absorption and Scattering of Light by Small Particles; Wiley: New York, 1983.
(B22) Kreibig, U.; Vollme, M. Optical properties of metal clusters; Springer: Berlin, 1995, and references therein.
(B23) Mie, G. Ann. Phys. 1908, 25, 377-445.
(B24) Huang, T.; Nallathamby, P. D.; Gillet, D.; Xu, X. H. Anal. Chem. 2007, 79, 7708-7718.
(B25) Kyriacou, S. V.; Brownlow, W. J.; Xu, X. H. Biochemistry 2004, 43, 140-147.
(B26) Lee, K. J.; Nallathamby, P. D.; Browning, L. M.; Osgood, C. J.; Xu, X.-H. N. ACS Nano 2007, 1, 133-143.
(B27) Nallathamby, P. D.; Lee, K. J.; Xu, X.-H. N. ACS Nano 2008, Vol. 2, No. 7, 1371-1380.
(B28) Xu, X.-H. N.; Brownlow, W. J.; Kyriacou, S. V.; Wan, Q.; Viola, J. J. Biochemistry 2004, 43, 10400-10413.
(B29) Xu, X.-H. N.; Chen, J.; Jeffers, R. B.; Kyriacou, S. V. Nano Letters 2002, 2, 175-182.
(B30) Metraux, G. S.; Mirkin, C. A. Adv. Mater. 2005, 17, 412-415.
(B31) Wiley, B.; Sun, Y.; Xia, Y. Acc. Chem. Res. 2007, 40, 1067-1076, and references therein.
(B32) Xu, X.-H. N.; Brownlow, W. J.; Huang, S.; Chen, J. Biochem. Biophys. Res. Commun. 2003, 305, 79-86.
(B33) Hubenthal, F. Progress in Surface Science 2007, 82, 378-387.
(B34) Liu, Y.; Zhang, W.; Yu, X.; Zhang, H.; Zhao, R.; Shangguan, D.; Li, Y.; Shen, B.; Liu, G. Sensors and Actuators B 2004, 99, 416-424.
(B35) Heinisch, R.; Zanetti, C.; Comin, F.; Fernandes, J.; Ramires, J.; Serrano Jr, C. Vascular Health and Risk Management 2005, 1, 245-250.
(B36) Skoog, T.; Dichtl, W.; Boquist, S.; Skoglund-Andersson, C.; Karpe, F.; Tang, R.; Bond, M.; Faire, U.; Nilsson, J.; Eriksson, P.; Hamsten, A. European Heart Journal 2002, 23, 376-383.
(B37) Hövel, H.; Fritz, S.; Hilger, A.; Kreibig, U.; Vollmer, M. Phys. Rev. B 1993, 48, 18178-18188.

1. A composition comprising:
at least one nanoparticle comprising a surface functionalized with at least one first monolayer component and at least one second monolayer component different from the first, wherein the molar ratio of the amount of the first monolayer component to the amount of the second monolayer component is adapted to provide a stability against aggregation of nanoparticles when a collection of the nanoparticles is dispersed in water.

2. The composition of claim 1, wherein the molar ratio of the amount of the first monolayer component to the amount of the second monolayer component is adapted to provide a stability of nanoparticles against their aggregation of at least seven days when a collection of the nanoparticles is dispersed in water, as measured by sizes and colors (spectra) of nanoparticles.

3. The composition of claim 1, wherein the molar ratio of the amount of the first monolayer component to the amount of the second monolayer component is adapted to provide a stability against aggregation of nanoparticles of at least 30 days when a collection of the nanoparticles is dispersed in water, as measured by sizes and colors (spectra) of nanoparticles.

4. The composition of claim 1, wherein the molar ratio of the amount of the first monolayer component to the amount of the second monolayer component is more than about 1:1 to about 1:20.

5. (canceled)

6. The composition of claim 1, wherein the first and second monolayer components are mercapto compounds.

7. The composition of claim 1, wherein the first and second monolayer components are mercapto compounds which each can be represented by A-B—C, wherein A is a functional group which binds to the nanoparticle surface, C is a reactive or unreactive group, and B is a linking group.

8. (canceled)

9. The composition of claim 1, wherein the first monolayer component is adapted to bind to a biological moiety, and the first and second monolayer components are molecules, and the first monolayer component is longer than the second monolayer component.

10. The composition of claim 1, wherein the nanoparticle further comprises a third component at the surface.

11. The composition of claim 1, wherein the nanoparticle further comprises a third component at the surface which provides electrostatic charge to the surface.

12. (canceled)

13. The composition of claim 1, wherein the composition comprises the nanoparticles having an average particle size of about 1 nm to about 100 mm.

14. (canceled)

15. The composition of claim 1, wherein the first component is further functionalized with a biological moiety.

16-19. (canceled)

20. The composition of claim 1, wherein the first component is further functionalized with a biological moiety at an average ratio of about one nanoparticle to about 0.5 to about 1.5 biological moiety.

21. The composition of claim 1, wherein the nanoparticle is a metal nanoparticle.

22. The composition of claim 1, wherein the nanoparticle is a noble metal nanoparticle.

23. The composition of claim 1, wherein the nanoparticle is a silver nanoparticle.

24. (canceled)

25. The composition of claim 1, wherein the nanoparticle is dispersible in water.

26. The composition of claim 1, wherein the nanoparticle is not a fluorescent nanoparticle.

27. The composition of claim 1, wherein the nanoparticle is a noble metal nanoparticle, wherein the first and second monolayer components are mercapto compounds, and a third component is present, and the composition comprises the nanoparticles having an average particle size of about 1 nm to about 100 nm.

28. (canceled)

29. The composition of claim 1, wherein the nanoparticle is a noble metal nanoparticle dispersible in water, wherein the first component is further functionalized with a biological moiety and a third component is present, and the nanoparticle has a particle size of about 1 nm to about 100 nm, and wherein the molar ratio of the amount of the first monolayer component to the amount of the second monolayer component is adapted to provide a stability against aggregation when a collection of the nanoparticles is dispersed in water.

30. (canceled)

31. A composition comprising:
at least one noble metal nanoparticle comprising a surface functionalized with at least one first monolayer component and at least one second monolayer component different from the first, wherein the first monolayer component is further functionalized with a biological moiety, and wherein the molar ratio of the amount of the first monolayer component to the amount of the second monolayer component is adapted to provide a stability against aggregation of nanoparticles when a collection of the nanoparticles is dispersed in water.

32-50. (canceled)

51. A composition comprising:
at least one nanoparticle comprising a surface functionalized with at least one first monolayer component and at least one second monolayer component different from the first, wherein the molar ratio of the amount of the first monolayer component to the amount of the second monolayer component is adapted to provide a stability against aggregation of nanoparticles when a collection of the nanoparticles is dispersed in water, wherein the first component is further functionalized with a biological moiety for binding to an analyte.

52-54. (canceled)

55. The composition of claim 51, wherein the molar ratio of the amount of the first monolayer component to the amount of the second monolayer component is more than about 1:1 to about 1:5.

56-57. (canceled)

58. The composition of claim 51, wherein the first and second monolayer components are mercapto compounds which each can be represented by A-B—C, wherein A is a functional group which binds to the nanoparticle surface, C is a reactive, and B is a linking group.

59-60. (canceled)

61. The composition of claim 51, wherein the nanoparticle further comprises a third component at the surface, which provides electrostatic charge to the surface.

62. (canceled)

63. The composition of claim 51, wherein the composition comprises the nanoparticles having an average particle size of about 1 nm to about 100 mm.

64. (canceled)

65. The composition of claim 51, wherein the biological moiety is a protein biological moiety, an antibody biological moiety, a monoclonal antibody biological moiety, or combinations thereof.

66-68. (canceled)

69. The composition of claim 51, wherein the first component is further functionalized with a biological moiety at an average ratio of about one nanoparticle to about 0.5 to about 1.5 biological moiety.

70. The composition of claim 51, wherein the nanoparticle is a noble metal nanoparticle.

71. A composition of claim 51, wherein the analyte is a peptide, protein, nucleic acid, drug, bio-mimetic, antibody, cell receptor, or combinations thereof.

72-76. (canceled)

77. composition of claim 51, wherein the analyte is an antibody, and the antibody is humanized chimeric, monoclonal, or combinations thereof.

78-80. (canceled)

81. A method comprising:
providing at least one noble metal nanoparticle,
functionalizing the surface of the noble metal nanoparticle with a first monolayer component and a second monolayer component different from the first.

82. The method of claim 81, wherein the nanoparticle is characterized by a particle size of about 1 nm to about 20 nm.

83. (canceled)

84. The method of claim 81, wherein the molar ratio of the amount of the first monolayer component to the amount of the second monolayer component is adapted to provide a stability against aggregation of nanoparticles when a collection of the nanoparticles is dispersed in water.

85-87. (canceled)

88. The method of claim 81, wherein the first and second monolayer components are mercapto compounds.

89-91. (canceled)

92. The method of claim 81, wherein the providing step comprises forming the nanoparticle so that the nanoparticle comprises a third monolayer surface component, and the functionalizing step comprises replacing at least some of the third monolayer surface component.

93-95. (canceled)

96. The method of claim 81, wherein the composition comprises the nanoparticles having an average particle size of about 1 nm to about 100 nm.

97-98. (canceled)

99. The method of claim 81, wherein the functionalized nanoparticle is dispersible in water.

100. The method of claim 81, wherein the functionalized nanoparticle is not a fluorescent nanoparticle.

101. A method comprising:
providing a composition according to claim 51, and
binding the composition to an analyte.

102. The method of claim 101, wherein the analyte is bound to a surface.

103. The method of claim 101, wherein the analyte is in solution.

104-105. (canceled)

106. The method of claim 101, wherein the analyte is a protein, an antibody, a drug, or combinations thereof.

107-108. (canceled)

109. The method of claim 101, further comprising imaging the bound nanoparticle with real time imaging.

110. The method of claim 101, further comprising detecting the bound nanoparticle under conditions of single molecule detection.

111-112. (canceled)

113. The method of claim 101, wherein the binding is carried out in vivo or in vitro.

114. (canceled)

115. The method of claim 101, wherein the binding is a specific binding.

116-120. (canceled)

121. A method of detecting an analyte using a sensor comprising a sensor molecule coupled to a nanoparticle, the method comprising:
exposing the sensor to the analyte, whereby the sensor molecule selectively binds to the analyte; and
detecting an optical property of the nanoparticle in real-time with single-molecule resolution.

122. The method of claim 121, wherein detecting an optical property of the nanoparticle comprises measuring the localized surface plasmon resonance spectrum of the nanoparticle.

123. The method of claim 121, wherein the sensor molecule comprises a protein or protein fragment and the analyte comprises a protein on a biological cell.

124. The method of claim 123, wherein at least one of the binding kinetics and binding affinity of the protein on the biological cell is measured in real-time.

125. (canceled)

126. The method of claim 122, wherein a sensor molecule is coupled to the nanoparticle through a mixed monolayer disposed on the surface of the nanoparticle, wherein the mixed monolayer comprises a set of first functional molecules and a set of second functional molecules that differ from the first functional molecules.

127. A complex comprising a protein or protein fragment coupled to a nanoparticle, wherein the protein or protein fragment is bound to a protein or protein fragment on the surface of a biological cell, thereby coupling the nanoparticle to the biological cell; and further wherein the nanoparticle exhibits localized surface plasmon resonance.

128. The complex of claim 127, wherein the protein or protein fragment is coupled to the nanoparticle through a mixed monolayer disposed on the surface of the nanoparticle and further wherein the mixed monolayer comprises a set of first functional molecules and a set of second functional molecules that differ from the first functional molecules.

129. The complex of claim 128, wherein the first functional molecules comprise a functional group capable of forming a covalent attachment to the sensor molecule.

130. The complex of claim 129, wherein the ratio of the first functional molecules to the second functional molecules in the monolayer is from about 1:1 to about 1:9.

131. The complex of claim 128, wherein the first and second functional molecules comprises alkanethiols.

132-133. (canceled)

134. The complex of claim 128, wherein the nanoparticle is a noble metal nanoparticle.

135-136. (canceled)
